(19) United States
(12) Patent Application Publication
(10) Pub. No.: US 2016/0260906 A1
(43) Pub. Date: Sep. 8, 2016

(54) ORGANIC LIGHT EMITTING COMPOUND AND ORGANIC LIGHT EMITTING DEVICE COMPRISING THE SAME

(71) Applicant: SFC CO., LTD., Chungcheongbuk-do (KR)

(72) Inventors: Bong-ki SHIN, Jeollabuk-do (KR); Bu-Bae PARK, Daegu (KR); Ji-hee PARK, Gyeongsangnam-do (KR); Bong-hyang LEE, Busan (KR); Kyung-Hwa PARK, Gangwon-do (KR)

(21) Appl. No.: 15/028,234

(22) PCT Filed: Oct. 10, 2014
(86) PCT No.: PCT/KR2014/009507

§ 371 Date: Apr. 8, 2016

(30) Foreign Application Priority Data
Oct. 11, 2013 (KR) 10-2013-0120942

(51) Int. Cl. H01L 51/00; C07D 401/14; C07D 403/14; C09K 11/02; C09K 11/06

(52) U.S. Cl. CPC H01L 51/0072; C09K 11/025; C09K 11/06; H01L 51/0085; C07D 403/14; H01L 51/0067; C07D 401/14; H01L 51/5012

(57) ABSTRACT

The present invention relates to an organic light emitting compound and an organic light emitting device comprising the same, and the organic light emitting device according to the present invention has excellent light emission efficiency and can be driven at low voltages, and thus have power efficiency and long lifespan characteristics.

### TECHNICAL FIELD

The present invention relates to organic light emitting compounds and organic electroluminescence devices including the same.

### BACKGROUND ART

Organic electroluminescence devices are devices in which when charges are injected into an organic light emitting layer disposed between an electron injecting electrode (cathode) and a hole injecting electrode (anode), electrons and holes combine with each other in the light emitting layer and then the electron-hole pairs decay to emit light. Organic electroluminescence devices can be fabricated even on flexible transparent substrates, such as plastic substrates. Other advantages of organic electroluminescence devices are low driving voltages of 10 V or less, relatively low power consumption, and accurate color representation compared to plasma display panels and inorganic electroluminescence displays. In addition, organic electroluminescence devices can represent green, blue, and red colors. Due to these advantages, organic electroluminescence devices have been the subject of intense interest as next-generation full-color display devices.

Luminescent materials are the most important factors determining the luminous efficiency of organic electroluminescence devices. Fluorescent materials are widely used at present as luminescent materials but the development of phosphorescent materials is theoretically considered an approach to further improve the luminous efficiency of organic electroluminescence devices in view of the mechanism of light emission. Thus, various phosphorescent materials have been developed and are currently being developed. Particularly, 4,4′-N,N′-dicarbazolebiphenyl (CBP) is most widely known as a phosphorescent host material. Organic electroluminescence devices are known that use, as hosts, carbazole compounds whose carbazole skeletons are substituted with various groups (Japanese Patent Publication Nos. 2008-214244 and 2003-133075) or BALq derivatives.

Organic electroluminescence devices using phosphorescent materials have considerably high current efficiency compared to devices using fluorescent materials. However, organic electroluminescence devices using BAlq and CBP as phosphorescent host materials do not offer significant advantages in terms of power efficiency over devices using fluorescent materials because of their higher driving voltages and do not reach a satisfactory level in terms of device life. Under these circumstances, there is a need to develop a more stable high-performance host material.

### DETAILED DESCRIPTION OF THE INVENTION

### Problems to be Solved by the Invention

Therefore, the present invention is intended to provide organic light emitting compounds that have improved power efficiency and life characteristics as well as high luminous efficiency compared to conventional luminescent materials. The present invention is also intended to provide organic electroluminescence devices that employ the organic light emitting compounds as luminescent materials, achieving low-voltage driving, high efficiency, and improved life characteristics.

### Means for Solving the Problems

Aspects of the present invention provide organic light emitting compounds represented by Formula 1:

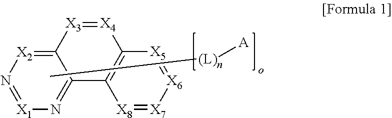

wherein X₁ to X₈, L, n, and o are as defined below and A is represented by Formula A1 or A2:

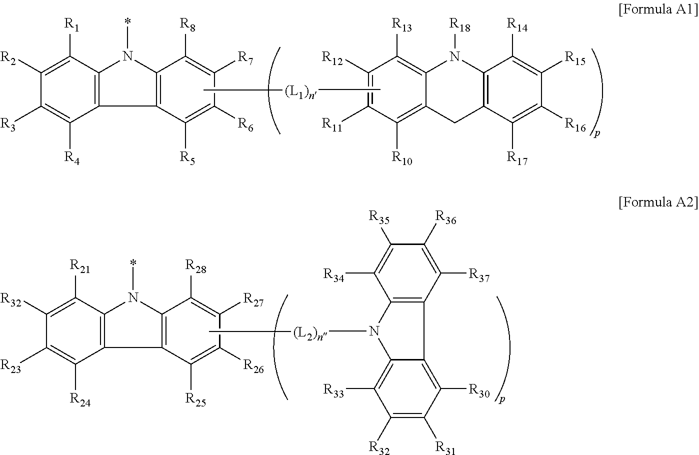

wherein each asterisk (*) represents a site at which the nitrogen atom is bonded to L and R₁ to R₈, R₁₀ to R₁₈, R₂₁ to R₂₈, R₃₀ to R₃₇, L₁, L₂, Y, m, n′, n″, and p are as defined below, and an organic electroluminescence device including at least one of the organic light emitting compounds.

### Effects of the Invention

The organic electroluminescence devices employing the organic light emitting compounds according to the present invention can be driven at low voltages compared to conventional devices employing phosphorescent host materials. The low-voltage driving leads to high power efficiency while at the same time achieving improved luminous efficiency and life characteristics. Due to these advantages, the organic electroluminescence devices of the present invention are suitable for use in various displays and white lighting systems.

### BRIEF DESCRIPTION OF THE DRAWINGS

FIG. 1 is a conceptual view illustrating a multilayer organic electroluminescence device according to one embodiment of the present invention.

### BEST MODE FOR CARRYING OUT THE INVENTION

The present invention will now be described in more detail.

One aspect of the present invention is directed to organic light emitting compounds represented by Formula 1:

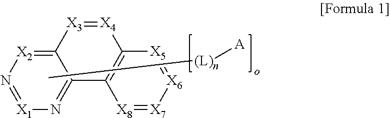

wherein X₁ to X8 are identical to or different from each other and are each independently N or CRₒ, provided that when CRₒ exists in plurality, the CRₒ groups may be identical to or different from each other, and A is represented by Formula A1 or A2:

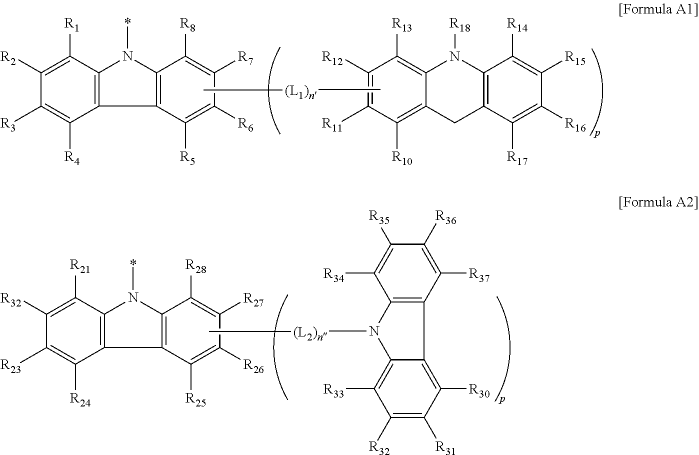

wherein each asterisk (*) represents a site at which the nitrogen atom is bonded to L, Rₒ, R₁ to R₈, R₁₀ to R₁₈, R₂₁ to R₂₈, and R₃₀ to R₃₇ are identical to or different from each other and are each independently selected from a hydrogen atom, a deuterium atom, substituted or unsubstituted C₁-C₃₀ alkyl groups, substituted or unsubstituted C₆-C₄₀ aryl groups, substituted or unsubstituted C₂-C₃₀ heteroaryl groups, halogen atoms, a hydroxyl group, a cyano group, a nitro group, an amino group, an amidino group, a hydrazino group, a hydrazono group, a carboxyl group or salts thereof, a sulfonic acid group or salts thereof, a phosphoric acid group or salts thereof, substituted or unsubstituted C₂-C₆₀ alkenyl groups, substituted or unsubstituted C₂-C₆₀ alkynyl groups, substituted or unsubstituted C₁-C₆₀ alkoxy groups, substituted or unsubstituted C₃-C₆₀ cycloalkyl groups, substituted or unsubstituted C₅-C₃₀ cycloalkenyl groups, substituted or unsubstituted C₅-C₆₀ aryloxy groups, substituted or unsubstituted C₁-C₃₀ alkylthioxy groups, substituted or unsubstituted C₅-C₃₀ arylthioxy groups, substituted or unsubstituted C₅-C₆₀ arylthio groups, —SiR₃₈R₃₉R₄₀, and —NR₄₁R₄₂, Rₒ, R₁ to R₈, R₁₀ to R₁₈, R₂₁ to R₂₈, R₃₀ to R₃₇, and substituents thereof may be bonded together to form a saturated or unsaturated ring, Y is a single bond, CR₅₁R₅₂, NR₅₃, O, S, Se, SiR₅₄R₅₅, GeR₅₆R₅₇, PR₅₈, PR₅₉(═O), C═O or BR₆₀, m is an integer of 1 or 2, provided that when m is 2, the plurality of Y groups may be identical to or different from each other, L, L₁, and L₂ are each independently a single bond or are each independently selected from substituted or unsubstituted C₁-C₆₀ alkylene groups, substituted or unsubstituted C₂-C₆₀ alkenylene groups, substituted or unsubstituted C₂-C₆₀ alkynylene groups, substituted or unsubstituted C₃-C₆₀ cycloalkylene groups, substituted or unsubstituted C₂-C₆₀ heterocycloalkylene groups, substituted or unsubstituted C₅-C₆₀ arylene groups, and substituted or unsubstituted C₂-C₆₀ heteroarylene groups, with the proviso that L, L₁, and L₂ may be each independently combined with an adjacent substituent to form a saturated or unsaturated ring, n, n′, and n″ are each independently an integer from 0 to 3, provided that when n, n′, and n″ are equal to or greater than 2, the plurality of L, L₁, and L₂ groups may be identical to or different from each other, respectively, o and p are each independently an integer from 1 to 3, and R₃₈ to R₄₂ and R₅₁ to R₆₀ have the same meanings as Rₒ, R₁ to R₈, R₁₀ to R₁₈, R₂₁ to R₂₈, and R₃₀ to R₃₇.

Specifically, L, L₁, and L₂ may be each independently selected from, but are not limited to, the following structures:

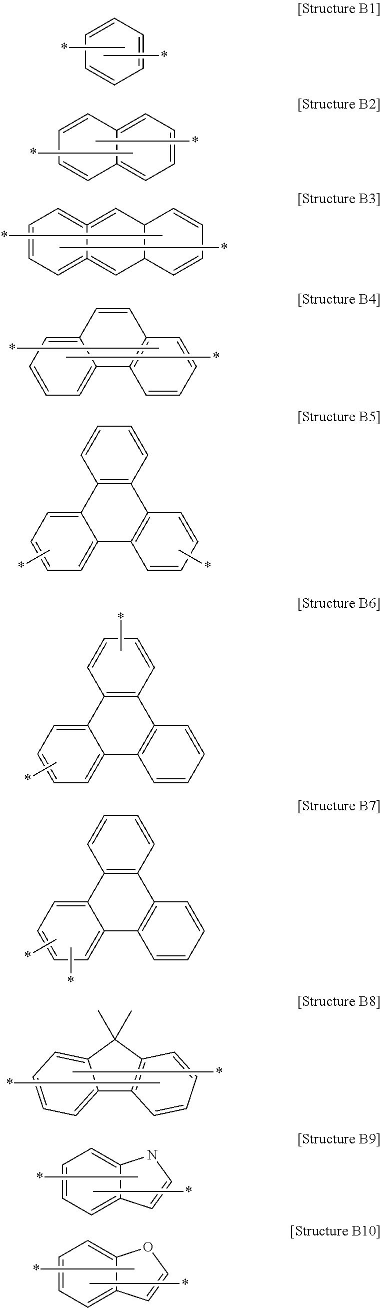

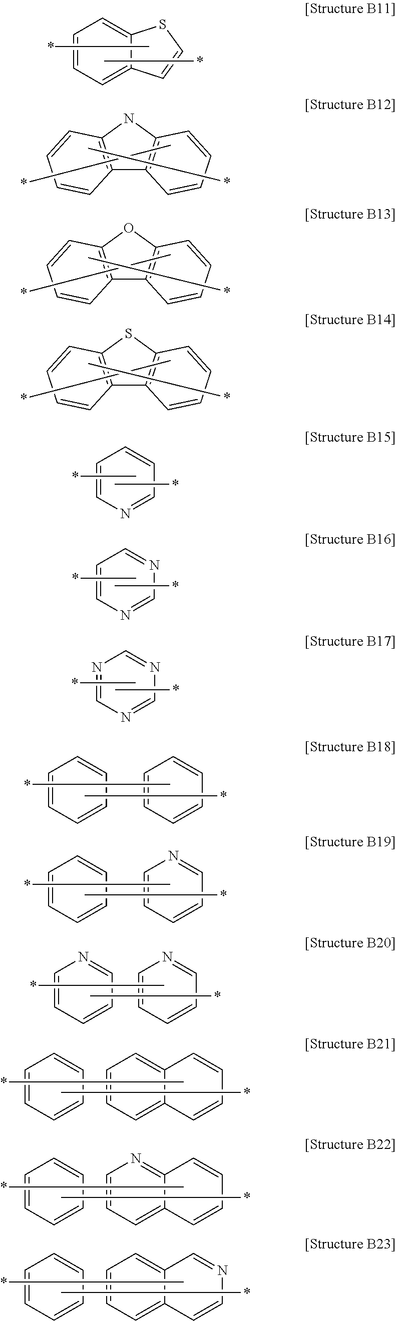

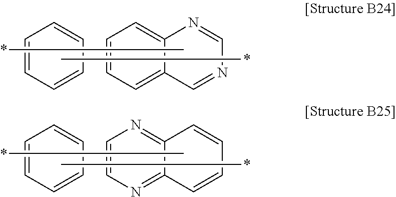

wherein hydrogen or deuterium atoms may be optionally bonded to the carbon atoms of the aromatic rings and R may optionally replace the nitrogen atoms, R having the same meaning as R₁ to R₈, R₁₀ to R₁₈, R₂₁ to R₂₈, and R₃₀ to R₃₇ defined in Formula 1.

The compounds of Formula 1 may vary in structure depending on the position where *-(L)ₙ—A is linked. The compounds of Formula 1 may be represented by Formula 1-1:

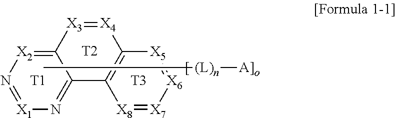

Specifically, the compounds of Formula 1 are represented by Formula 2 when *-(L)ₙ—A is linked to T1, Formula 3 when linked to T2, Formula 4 when linked to T1 and T2, Formula 5 when linked to T3, Formula 6 when linked to T2 and T3, Formula 7 when linked to T1 and T3 or Formula 8 when linked to T1, T2, and T3:

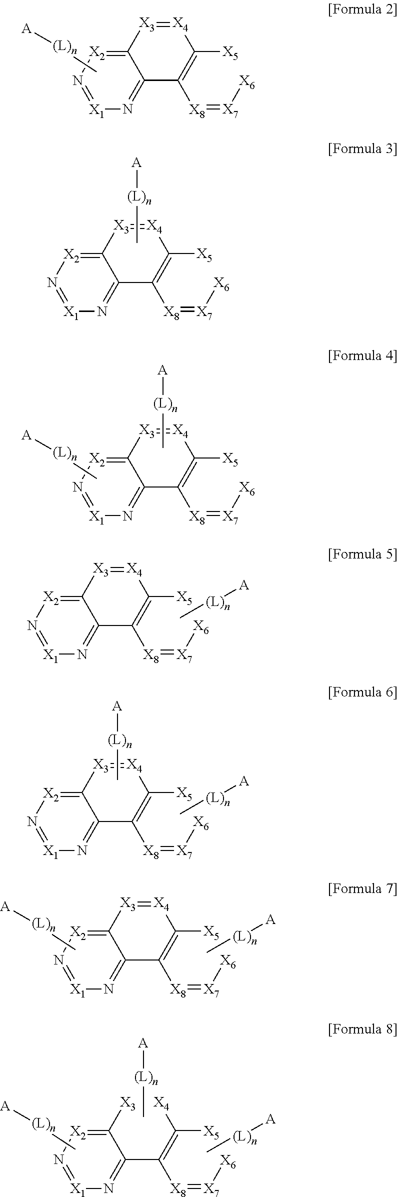

wherein L, X₁ to X₈, A, and n are as defined in Formula 1.

According to one preferred embodiment of the present invention, the organic light emitting compounds of Formula 1 may be more specifically selected from, but are not limited to, the following compounds 1 to 29:

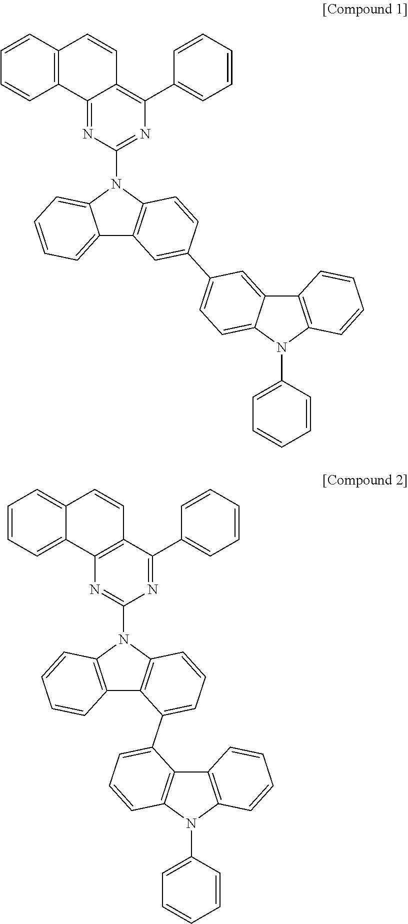 [SMILES: c1ccc(-c2nc(-n3c4ccccc4c4cc(-c5ccc6c(c5)c5ccccc5n6-c5ccccc5)ccc43)nc3c2ccc2ccccc23)cc1 ; c1ccc(-c2nc(-n3c4ccccc4c4c(-c5cccc6c5c5ccccc5n6-c5ccccc5)cccc43)nc3c2ccc2ccccc23)cc1]

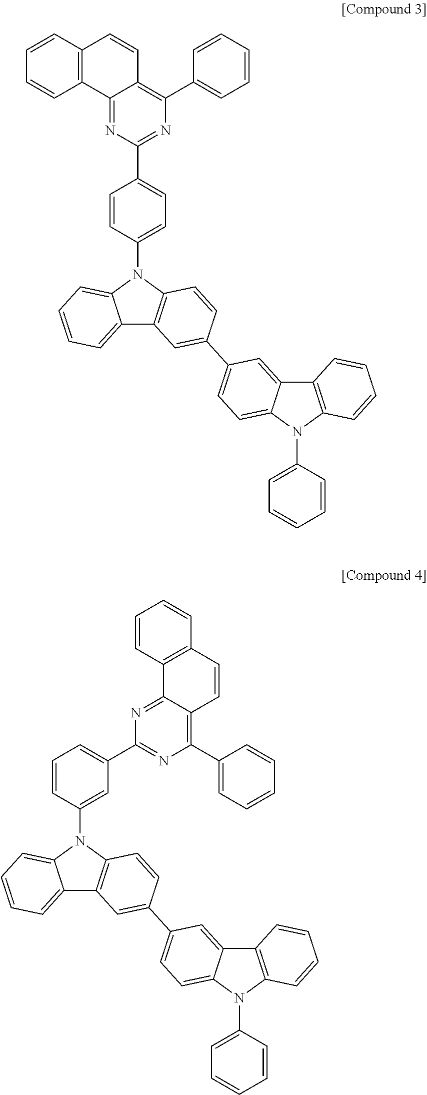 [SMILES: c1ccc(-c2nc(-c3ccc(-n4c5ccccc5c5cc(-c6ccc7c(c6)c6ccccc6n7-c6ccccc6)ccc54)cc3)nc3c2ccc2ccccc23)cc1 ; c1ccc(-c2nc(-c3cccc(-n4c5ccccc5c5cc(-c6ccc7c(c6)c6ccccc6n7-c6ccccc6)ccc54)c3)nc3c2ccc2ccccc23)cc1]

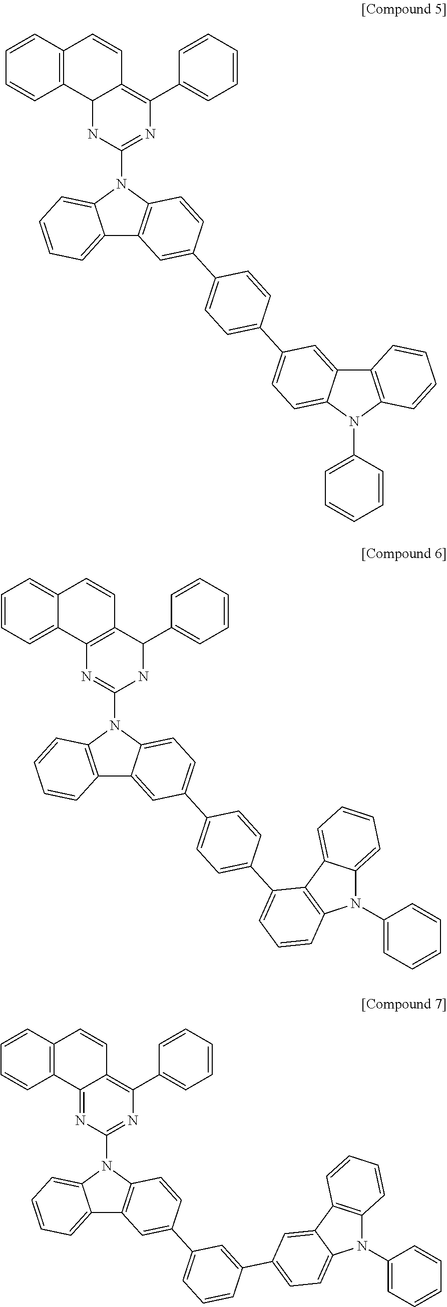 [SMILES: C1=Cc2ccccc2C2NC(n3c4ccccc4c4cc(-c5ccc(-c6ccc7c(c6)c6ccccc6n7-c6ccccc6)cc5)ccc43)=NC(c3ccccc3)=C12 ; c1ccc(C2NC(n3c4ccccc4c4cc(-c5ccc(-c6cccc7c6c6ccccc6n7-c6ccccc6)cc5)ccc43)=Nc3c2ccc2ccccc32)cc1 ; c1ccc(-c2nc(-n3c4ccccc4c4cc(-c5cccc(-c6ccc7c(c6)c6ccccc6n7-c6ccccc6)c5)ccc43)nc3c2ccc2ccccc23)cc1]

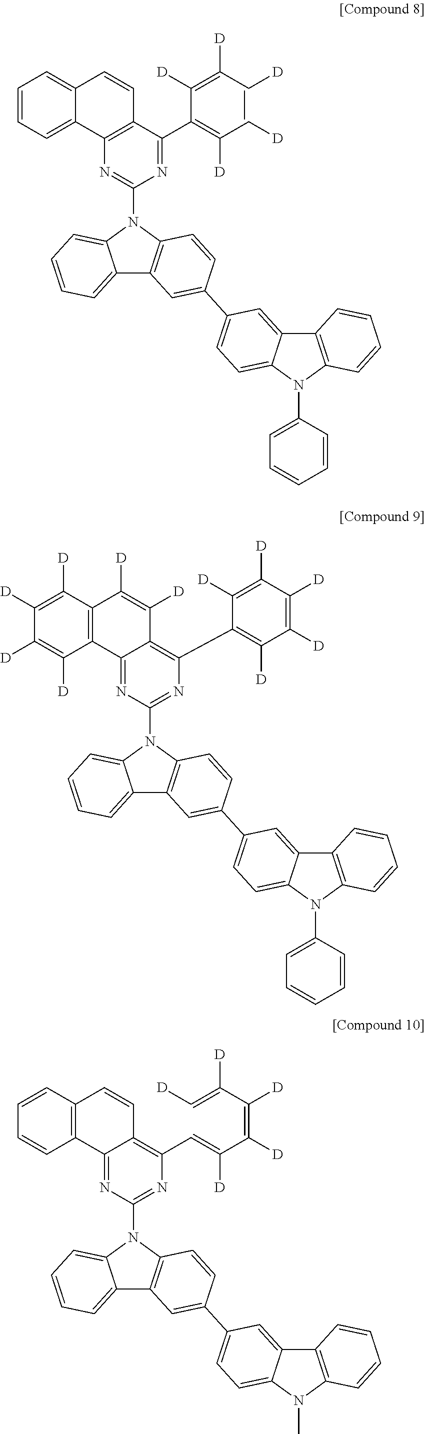 [SMILES: [2H]C/C([2H])=C([2H])\C(=C(\[2H])C[2H])c1nc(-n2c3ccccc3c3cc(-c4ccc5c(c4)c4ccccc4n5-c4ccccc4)ccc32)nc2c1ccc1ccccc12 ; [2H]c1c([2H])c([2H])c(-c2nc(-n3c4ccccc4c4cc(-c5ccc6c(c5)c5ccccc5n6-c5ccccc5)ccc43)nc3c2c([2H])c([2H])c2c([2H])c([2H])c([2H])c([2H])c23)c([2H])c1[2H] ; [2H]/C=C([2H])/C([2H])=C([2H])\C([2H])=C\c1nc(-n2c3ccccc3c3cc(-c4ccc5c(c4)c4ccccc4n5C)ccc32)nc2c1ccc1ccccc12]

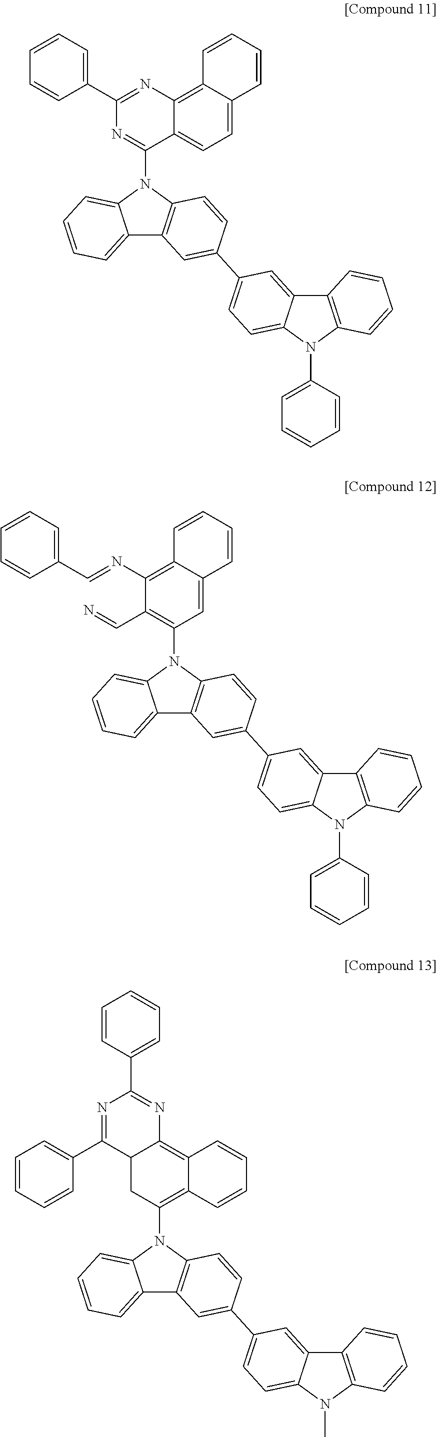 [SMILES: c1ccc(-c2nc(-n3c4ccccc4c4cc(-c5ccc6c(c5)c5ccccc5n6-c5ccccc5)ccc43)c3ccc4ccccc4c3n2)cc1 ; N=Cc1c(-n2c3ccccc3c3cc(-c4ccc5c(c4)c4ccccc4n5-c4ccccc4)ccc32)cc2ccccc2c1/N=C/c1ccccc1 ; Cn1c2ccccc2c2cc(-c3ccc4c(c3)c3ccccc3n4C3=c4ccccc4=C4N=C(c5ccccc5)N=C(c5ccccc5)C4C3)ccc21]

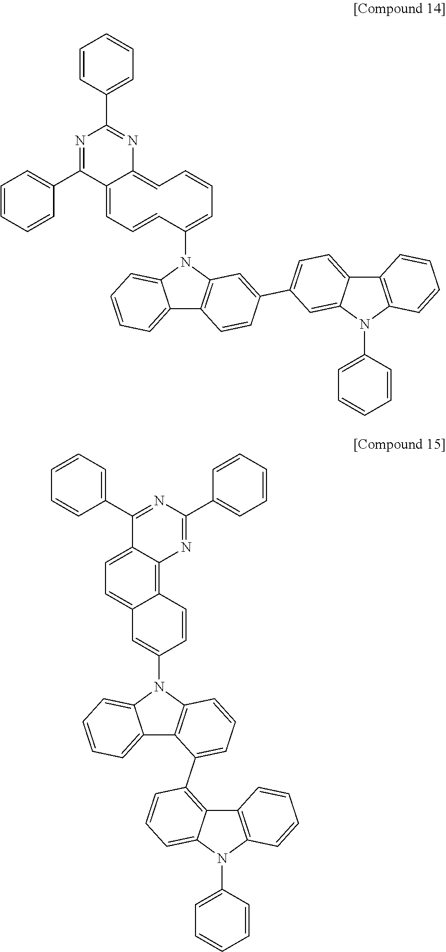 [SMILES: c1ccc(-c2nc(-c3ccccc3)c3cccc(-n4c5ccccc5c5ccc(-c6ccc7c8ccccc8n(-c8ccccc8)c7c6)cc54)ccccc3n2)cc1 ; c1ccc(-c2nc(-c3ccccc3)c3ccc4cc(-n5c6ccccc6c6c(-c7cccc8c7c7ccccc7n8-c7ccccc7)cccc65)ccc4c3n2)cc1]

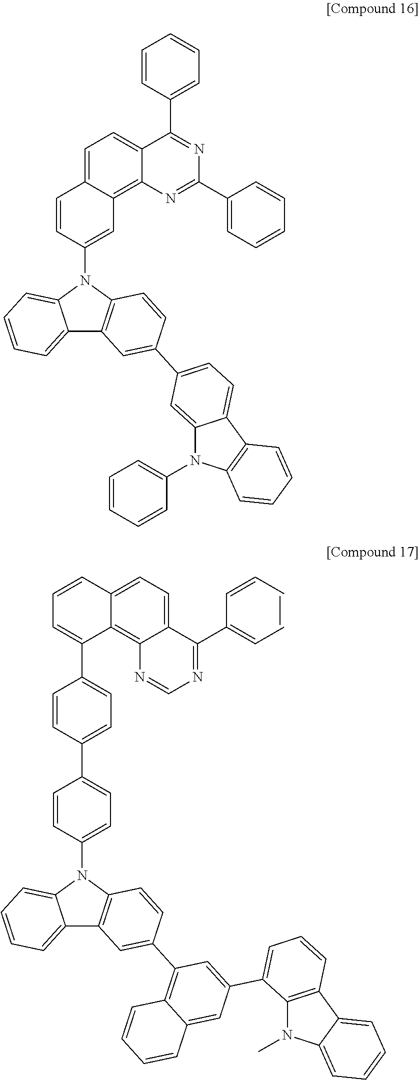 [SMILES: c1ccc(-c2nc(-c3ccccc3)c3ccc4ccc(-n5c6ccccc6c6cc(-c7ccc8c9ccccc9n(-c9ccccc9)c8c7)ccc65)cc4c3n2)cc1 ; C/C=C\C(=C/C)c1ncnc2c1ccc1cccc(-c3ccc(-c4ccc(-n5c6ccccc6c6cc(-c7cc(-c8cccc9c%10ccccc%10n(C)c89)cc8ccccc78)ccc65)cc4)cc3)c12]

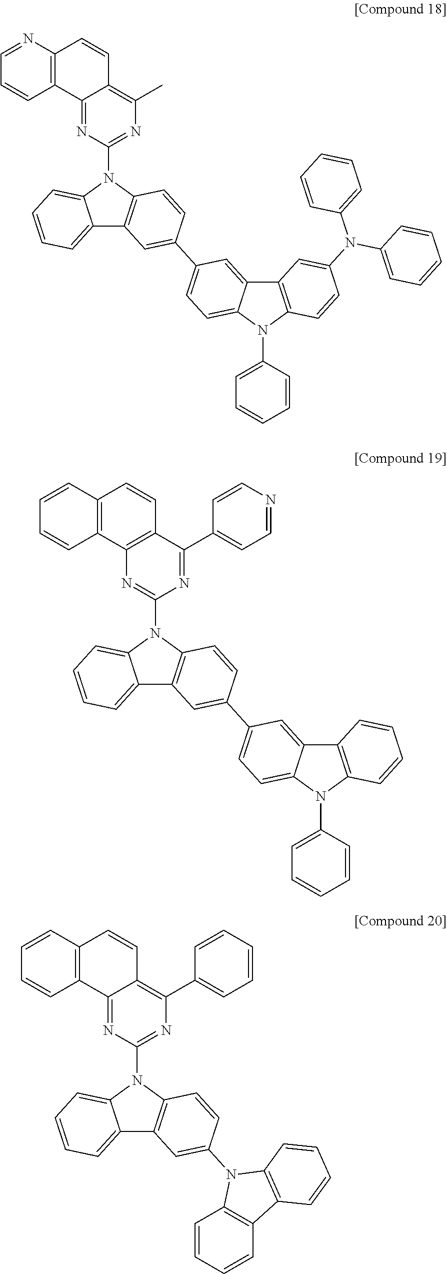 [SMILES: Cc1nc(-n2c3ccccc3c3cc(-c4ccc5c(c4)c4cc(N(c6ccccc6)c6ccccc6)ccc4n5-c4ccccc4)ccc32)nc2c1ccc1ncccc12 ; c1ccc(-n2c3ccccc3c3cc(-c4ccc5c(c4)c4ccccc4n5-c4nc(-c5ccncc5)c5ccc6ccccc6c5n4)ccc32)cc1 ; c1ccc(-c2nc(-n3c4ccccc4c4cc(-n5c6ccccc6c6ccccc65)ccc43)nc3c2ccc2ccccc23)cc1]

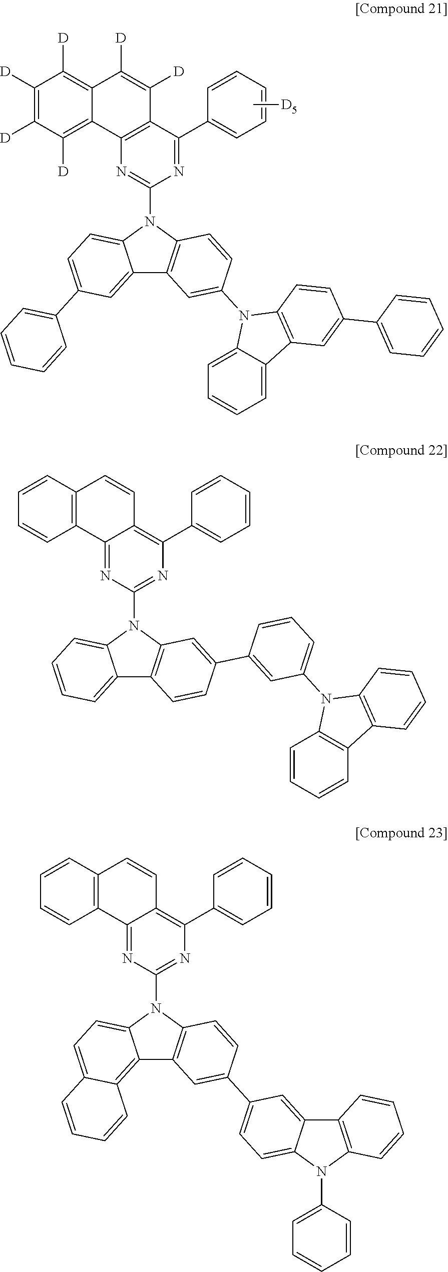

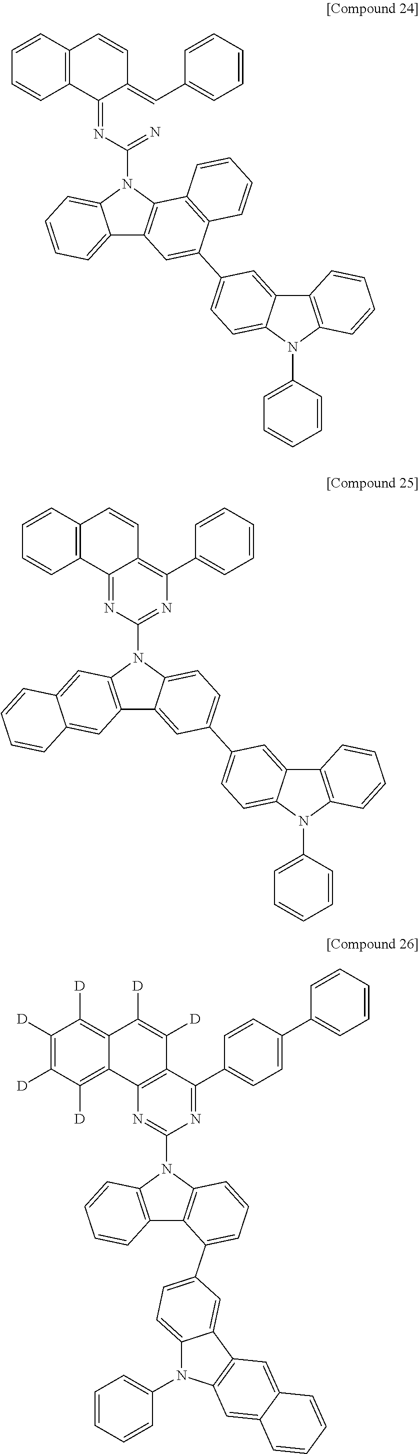 [SMILES: N=C(/N=C1\C(=C\c2ccccc2)C=Cc2ccccc21)n1c2ccccc2c2cc(-c3ccc4c(c3)c3ccccc3n4-c3ccccc3)c3ccccc3c21 ; c1ccc(-c2nc(-n3c4ccc(-c5ccc6c(c5)c5ccccc5n6-c5ccccc5)cc4c4cc5ccccc5cc43)nc3c2ccc2ccccc23)cc1 ; [2H]c1c([2H])c([2H])c2c(c1[2H])c([2H])c([2H])c1c(-c3ccc(-c4ccccc4)cc3)nc(-n3c4ccccc4c4c(-c5ccc6c(c5)c5cc7ccccc7cc5n6-c5ccccc5)cccc43)nc12]

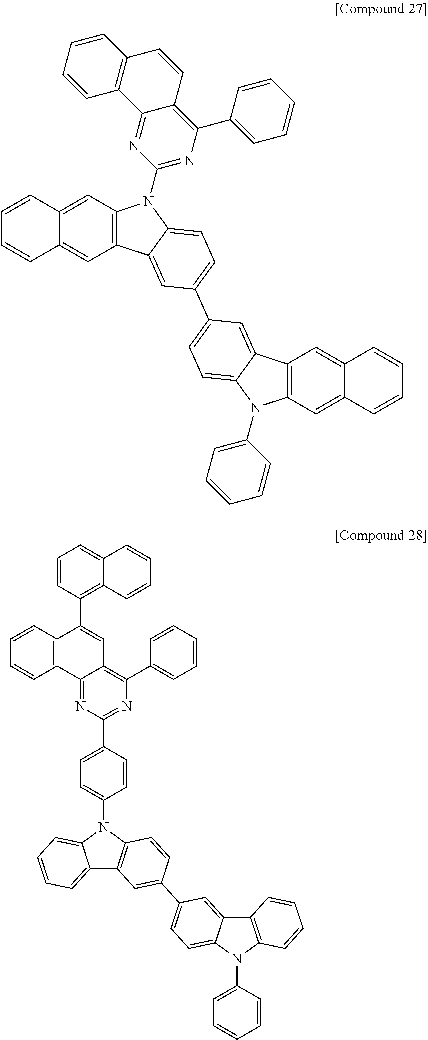 [SMILES: c1ccc(-c2nc(-n3c4ccc(-c5ccc6c(c5)c5cc7ccccc7cc5n6-c5ccccc5)cc4c4cc5ccccc5cc43)nc3c2ccc2ccccc23)cc1 ; C1=C\C/C(c2cccc3ccccc23)=C\c2c(nc(-c3ccc(-n4c5ccccc5c5cc(-c6ccc7c(c6)c6ccccc6n7-c6ccccc6)ccc54)cc3)nc2-c2ccccc2)C\C=C/1]

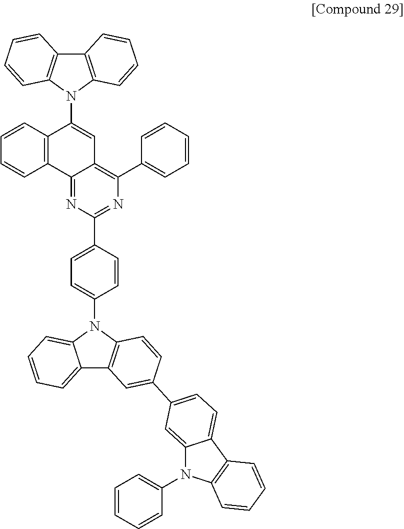 [SMILES: c1ccc(-c2nc(-c3ccc(-n4c5ccccc5c5cc(-c6ccc7c8ccccc8n(-c8ccccc8)c7c6)ccc54)cc3)nc3c2cc(-n2c4ccccc4c4ccccc42)c2ccccc23)cc1]

A further aspect of the present invention is directed to an organic electroluminescence device including a first electrode, a second electrode, and at least one organic layer interposed between the first and second electrodes wherein the organic layer includes at least one of the organic light emitting compounds represented by Formula 1.

The organic layer including the organic light emitting compound of the present invention may include at least one layer selected from a hole injecting layer, a hole transport layer, a functional layer having functions of both hole injection and hole transport, a light emitting layer, an electron transport layer, and an electron injecting layer.

The organic layer interposed between the first and second electrodes may include a light emitting layer. The light emitting layer may be composed of a host and a dopant. The organic light emitting compound of the present invention may be used as the host.

In the case where the light emitting layer includes a host and a dopant, the content of the dopant may be typically selected in the range of about 0.01 to about 20 parts by weight, based on 100 parts by weight the host.

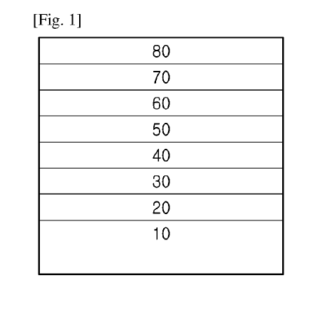

Hereinafter, the organic electroluminescence device of the present invention will be explained in more detail with reference to FIG. 1.

FIG. 1 is a cross-sectional view illustrating the structure of an organic electroluminescence device according to one embodiment of the present invention. The organic electroluminescence device includes an anode 20, a hole transport layer 40, an organic light emitting layer 50, an electron transport layer 60, and a cathode 80. The organic electroluminescence device may optionally further include a hole injecting layer 30 and an electron injecting layer 70. In addition to these layers, one or more intermediate layers may be further formed in the organic electroluminescence device. A hole blocking layer or an electron blocking layer may be further formed in the organic electroluminescence device. The device may further include one or more organic layers with various functions depending on the desired characteristics thereof.

Referring to FIG. 1, a detailed description is given of the organic electroluminescence device and its fabrication method.

First, an electrode material for the anode 20 is coated on a substrate 10 to form the anode 20. The substrate 10 may be any of those used in general organic electroluminescence devices. The substrate 10 is preferably an organic substrate or a transparent plastic substrate that is excellent in transparency, surface smoothness, ease of handling, and waterproofness. A highly transparent and conductive metal oxide, such as indium tin oxide (ITO), indium zinc oxide (IZO), tin oxide (SnO₂) or zinc oxide (ZnO), is used as the anode material.

A material for the hole injecting layer 30 is coated on the anode 20 by vacuum thermal evaporation or spin coating to form the hole injecting layer 30. Then, a material for the hole transport layer 40 is coated on the hole injecting layer 30 by vacuum thermal evaporation or spin coating to form the hole transport layer 40.

The material for the hole injecting layer is not specially limited so long as it is commonly used in the art, and specific examples thereof include 4,4′,4″-tris(2-naphthylphenyl-phenylamino)triphenylamine (2-TNATA), N,N′-di(1-naphthyl)-N,N′-diphenylbenzidine (NPD), N,N′-diphenyl-N,N′-bis(3-methylphenyl)-1,1′-biphenyl-4,4′-diamine (TPD), and N,N′-diphenyl-N,N′-bis[4-(phenyl-m-tolylamino)phenyl]biphenyl-4,4′-diamine (DNTPD).

The material for the hole transport layer is not specially limited so long as it is commonly used in the art, and examples thereof include N,N′-bis(3-methylphenyl)-N,N′-diphenyl-[1,1-biphenyl]-4,4′-diamine (TPD) and N,N′-di(naphthalen-1-yl)-N,N′-diphenylbenzidine (α-NPD).

Subsequently, the organic light emitting layer 50 is laminated on the hole transport layer 40. A hole blocking layer (not shown) may be optionally formed on the organic light emitting layer 50 by vacuum thermal evaporation or spin coating. The hole blocking layer blocks holes from entering the cathode through the organic light emitting layer. This role of the hole blocking layer prevents the life and efficiency of the device from deteriorating. A material having a very low highest occupied molecular orbital (HOMO) energy level is used for the hole blocking layer. The hole blocking material is not particularly limited so long as it has the ability to transport electrons and a higher ionization potential than the light emitting compound. Representative examples of suitable hole blocking materials include BAlq, BCP, and TPBI.

Examples of materials for the hole blocking layer include, but are not limited to, BAlq, BCP, Bphen, TPBI, NTAZ, BeBq₂, OXD-7, and Liq.

The electron transport layer 60 is deposited on the hole blocking layer by vacuum thermal evaporation or spin coating and the electron injecting layer 70 is formed thereon. A metal for the formation of the cathode is deposited on the electron injecting layer 70 by vacuum thermal evaporation to form the cathode 80, completing the fabrication of the organic EL device. As the metal for the formation of the cathode, there may be used, for example, lithium (Li), magnesium (Mg), aluminum (Al), aluminum-lithium (Al-Li), calcium (Ca), magnesium-indium (Mg-In) or magnesium-silver (Mg-Ag). The organic EL device may be of top emission type. In this case, a transmissive material, such as ITO or IZO, may be used to form the cathode.

The material for the electron transport layer functions to stably transport electrons injected from the electron injecting electrode (i.e. the cathode). The material for the electron transport layer may be any known electron transport material, and examples thereof include, but are not limited to, quinoline derivatives, particularly, tris(8-quinolinolate)aluminum (Alq3), TAZ, Balq, Bebq₂, and AND. Oxadiazole derivatives, such as PBD, BMD, and BND, may also be used.

The organic light emitting layer of the organic electroluminescence device may further include one or more phosphorescent dopants, in addition to at least one of the organic light emitting compounds represented by Formula 1.

The phosphorescent dopants employed in the organic electroluminescence device may be, for example, copper, boron, and metal complexes. Examples of the metal complexes include, but are not limited to, iridium, platinum, palladium, and ruthenium complexes.

The light emitting layer may further include one or more phosphorescent host compounds, in addition to at least one of the organic light emitting compounds represented by Formula 1.

One or more layers selected from the hole injecting layer, the hole transport layer, the electron blocking layer, the light emitting layer, the hole blocking layer, the electron transport layer, and the electron injecting layer may be formed by a monomolecular deposition or solution process. According to the deposition process, the material for each layer is evaporated under heat and vacuum or reduced pressure to form the layer in the form of a thin film. According to the solution process, the material for each layer is mixed with a suitable solvent and the mixture is then formed into a thin film by a suitable method, such as ink-jet printing, roll-to-roll coating, screen printing, spray coating, dip coating or spin coating.

The organic electroluminescence device can be used in systems selected from flat panel displays, flexible displays, monochromatic flat panel lighting systems, white flat panel lighting systems, flexible monochromatic lighting systems, and flexible white lighting systems.

### MODE FOR CARRYING OUT THE INVENTION

The present invention will be explained in more detail with reference to preferred embodiments in the following examples. However, it will be appreciated by those skilled in the art that these embodiments are provided for illustrative purposes only and are not intended to limit the scope of the invention.

### SYNTHESIS EXAMPLE 1

### Synthesis of Compound 2

[Reaction 1-1] Synthesis of Intermediate 1-a

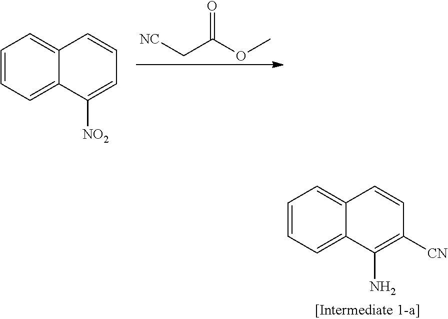

1-Nitronaphthalene (97 g, 0.56 mol), methyl cyanoacetate (166.5 g, 1.68 mol), potassium cyanide (40.1 g, 0.62 mol), potassium hydroxide (62.9 g, 1.12 mol), and 970 mL of dimethylformamide were mixed and stirred at 60° C. for 12 h. The mixture was concentrated under reduced pressure to remove the solvent, and then 500 mL of a 10% aqueous solution of sodium hydroxide was added thereto. The resulting mixture was refluxed for about 1 h. The reaction mixture was extracted with ethyl acetate, separated by column chromatography, and recrystallized from toluene and heptane, affording 50.8 g of Intermediate 1-a (yield 54%).

[Reaction 1-2] Synthesis of Intermediate 1-b

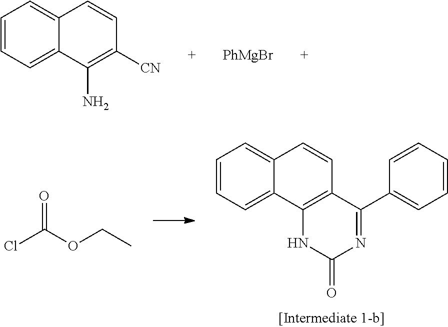

Intermediate 1-a (25.0 g, 149 mmol) synthesized in Reaction 1-1 was dissolved and stirred in 200 mL of tetrahydrofuran. To the solution was added dropwise phenylmagnesium bromide (3.0 M in Et₂O) (104 mL, 313 mmol). The mixture was refluxed at 0° C. for about 1 h. After dropwise addition of ethyl chloroformate (19.4 g, 179 mmol), the resulting mixture was refluxed for about 1 h. The reaction mixture was made weakly acidic by addition of an aqueous ammonium chloride solution and washed with water and heptane, affording 32.4 g of Intermediate 1-b (yield 80%).

[Reaction 1-3] Synthesis of Intermediate 1-c

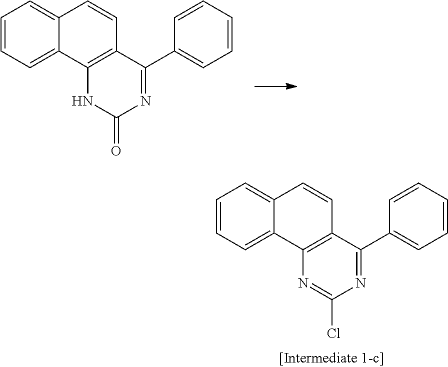 [SMILES: O=c1nc(-c2ccccc2)c2ccc3ccccc3c2[nH]1 ; Clc1nc(-c2ccccc2)c2ccc3ccccc3c2n1]

Intermediate 1-b (30.0 g, 110 mmol) synthesized in Reaction 1-2 was refluxed in about 150 mL of phosphorus oxychloride for 12 h. After cooling to −20° C., about 400 mL of distilled water was added dropwise. The reaction solution was filtered and the obtained solid was recrystallized from toluene and heptane, affording 14.1 g of Intermediate 1-c (yield 44%).

[Reaction 1-4] Synthesis of Intermediate 1-d

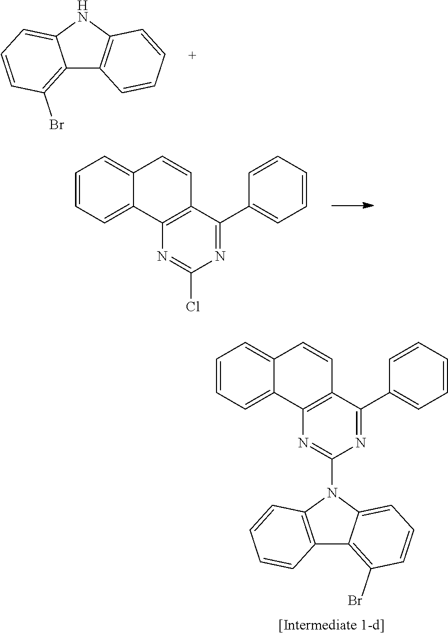 [SMILES: Brc1cccc2[nH]c3ccccc3c12 ; Clc1nc(-c2ccccc2)c2ccc3ccccc3c2n1 ; Brc1cccc2c1c1ccccc1n2-c1nc(-c2ccccc2)c2ccc3ccccc3c2n1]

60% sodium hydride (2.1 g, 53 mmol) and 50 mL of dimethylformamide were mixed and cooled to 0° C. To the mixture was added dropwise 4-bromo-9H-carbazole (10.0 g, 41 mmol) and 100 mL of dimethylformamide. The resulting mixture was stirred for about 1 h. A solution of Intermediate 1-c (15.4 g, 53 mmol) synthesized in Reaction 1-3 in 100 mL of dimethylformamide was added dropwise. The mixture was allowed to warm to room temperature, followed by stirring for 1 h. 600 mL of distilled water was added to precipitate a solid. The solid was collected by filtration and recrystallized from toluene, affording 16.2 g of Intermediate 1-d (yield 79%).

[Reaction 1-5] Synthesis of Intermediate 1-e

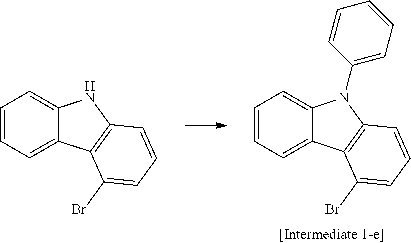 [SMILES: Brc1cccc2[nH]c3ccccc3c12 ; Brc1cccc2c1c1ccccc1n2-c1ccccc1]

A mixture of 4-bromo-9H-carbazole (20.0 g, 81.3 mmol), iodobenzene (33.2 g, 162.5 mmol), copper powder (10.3 g, 162.5 mmol), 18-crown-6 (4.3 g, 16.3 mmol), and potassium carbonate (33.7 g, 243.9 mmol) were refluxed in 200 mL of 1,2-dichlorobenzene at 180° C. for 24 h. The reaction mixture was filtered at a high temperature and separated by column chromatography, affording 21.0 g of Intermediate 1-e (yield 80%).

[Reaction 1-6] Synthesis of Intermediate 1-f

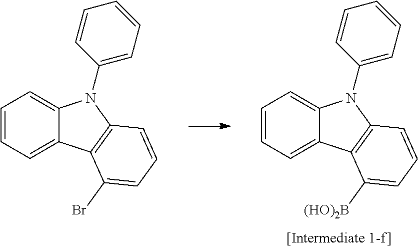 [SMILES: Brc1cccc2c1c1ccccc1n2-c1ccccc1 ; OB(O)c1cccc2c1c1ccccc1n2-c1ccccc1]

Intermediate 1-e (21.0 g, 65 mmol) synthesized in Reaction 1-5 was stirred in 210 mL of tetrahydrofuran at −78° C. After dropwise addition of 1.6 M n-butyllithium (49 mL, 78 mmol), stirring was continued for 1 h while maintaining the temperature at −78° C. Trimethylborate (8.1 g, 78 mmol) was slowly added dropwise. The resulting mixture was allowed to warm to room temperature, followed by stirring for 2 h. 100 mL of a 2 M aqueous solution of hydrochloric acid was added at room temperature and stirring was continued for 30 min. The reaction mixture was extracted with ethyl acetate and recrystallized from dichloromethane and hexane, affording 12.5 g of Intermediate 1-f (yield 67%).

[Reaction 1-7] Synthesis of Compound 2

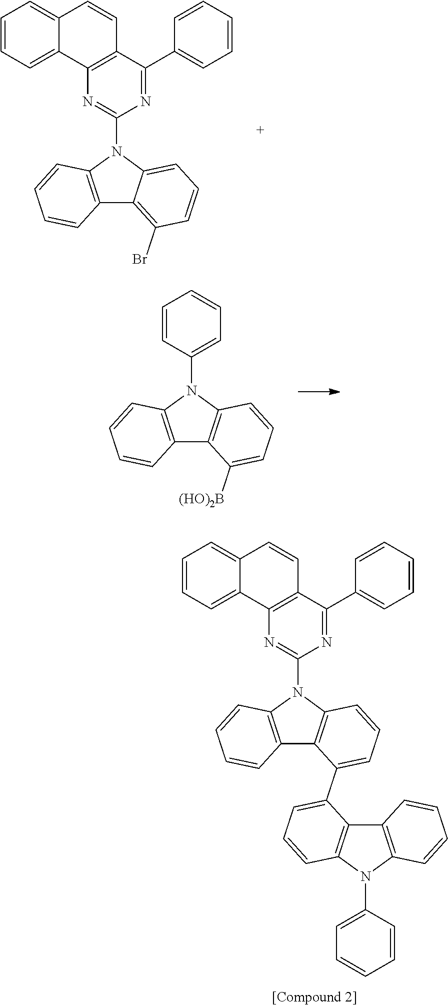

A mixture of Intermediate 1-d (16 g, 32 mmol) synthesized in Reaction 1-4, Intermediate 1-f (11.2 g, 39 mmol) synthesized in Reaction 1-6, tetrakis(triphenylphosphine)palladium (0.7 g, 0.64 mmol), and potassium carbonate (8.9 g, 64 mmol) in 80 mL of 1,4-dioxane, 80 mL of toluene, and 30 mL of distilled water was refluxed for 12 h. The reaction mixture was extracted with ethyl acetate and recrystallized from dichloromethane and acetone, affording 7.2 g of Compound 2 (yield 34%).

MS: m/z 663

### SYNTHESIS EXAMPLE 2

### Synthesis of Compound 8

[Reaction 2-1] Synthesis of Intermediate 2-a

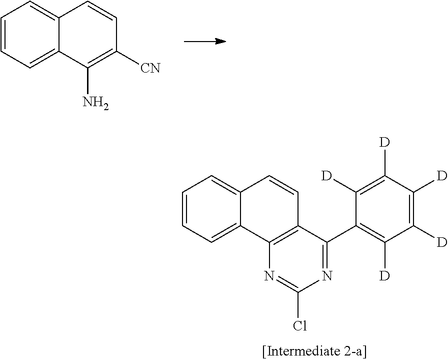 [SMILES: N#Cc1ccc2ccccc2c1N ; [2H]c1c([2H])c([2H])c(-c2nc(Cl)nc3c2ccc2ccccc23)c([2H])c1[2H]]

Intermediate 2-a (yield 45%) was synthesized in the same manner as in Reactions 1-2 and 1-3, except that pentadeuterophenylmagnesium bromide was used instead of phenylmagnesium bromide in Reaction 1-2.

[Reaction 2-2] Synthesis of Intermediate 2-b

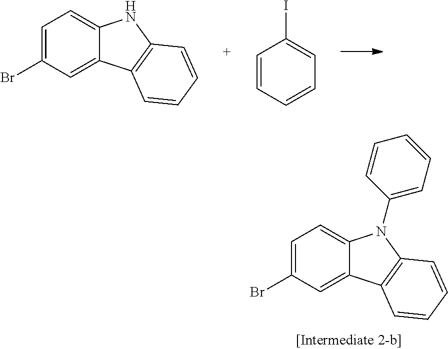 [SMILES: Brc1ccc2[nH]c3ccccc3c2c1 ; Ic1ccccc1 ; Brc1ccc2c(c1)c1ccccc1n2-c1ccccc1]

Intermediate 2-b (yield 86%) was synthesized in the same manner as in Reaction 1-5, except that 3-bromo-9H-carbazole was used instead of 4-bromo-9H-carbazole.

[Reaction 2-3] Synthesis of Intermediate 2-c

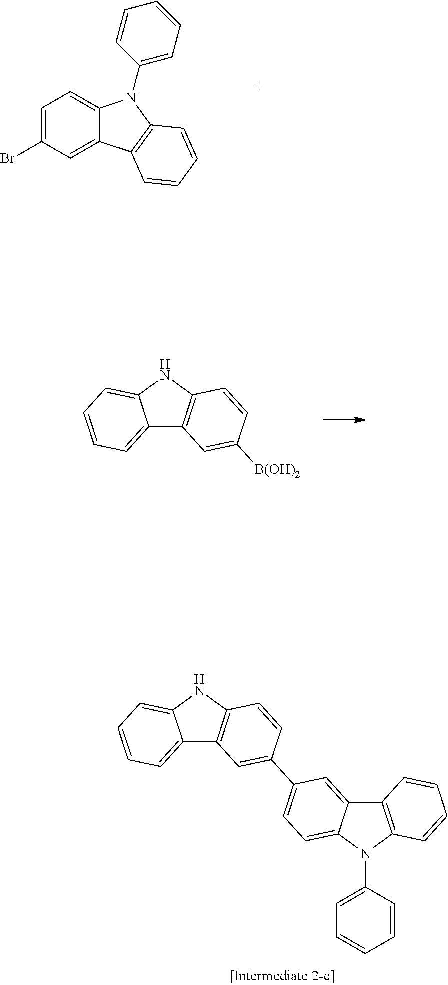 [SMILES: Brc1ccc2c(c1)c1ccccc1n2-c1ccccc1 ; OB(O)c1ccc2[nH]c3ccccc3c2c1 ; c1ccc(-n2c3ccccc3c3cc(-c4ccc5[nH]c6ccccc6c5c4)ccc32)cc1]

Intermediate 2-c (yield 73%) was synthesized in the same manner as in Reaction 1-7, except that Intermediate 2-b synthesized in Reaction 2-2 and 9H-carbazol-3-ylboronic acid were used instead of Intermediate 1-d and Intermediate 1-f, respectively.

[Reaction 2-4] Synthesis of Compound 8

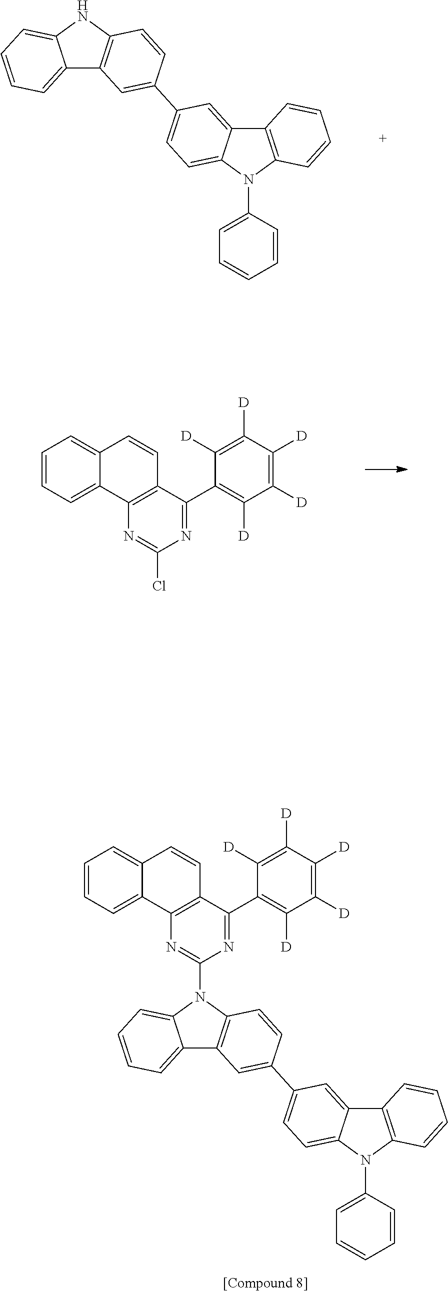 [SMILES: c1ccc(-n2c3ccccc3c3cc(-c4ccc5[nH]c6ccccc6c5c4)ccc32)cc1 ; [2H]c1c([2H])c([2H])c(-c2nc(Cl)nc3c2ccc2ccccc23)c([2H])c1[2H] ; [2H]c1c([2H])c([2H])c(-c2nc(-n3c4ccccc4c4cc(-c5ccc6c(c5)c5ccccc5n6-c5ccccc5)ccc43)nc3c2ccc2ccccc23)c([2H])c1[2H]]

Compound 8 (yield 32%) was synthesized in the same manner as in Reaction 1-4, except that Intermediate 2-c synthesized in Reaction 2-3 and Intermediate 2-a synthesized in Reaction 2-1 were used instead of 4-bromo-9H-carbazole and Intermediate 1-c, respectively.

MS: m/z 668

### SYNTHESIS EXAMPLE 3

### Synthesis of Compound 26

[Reaction 3-1] Synthesis of Intermediate 3-a

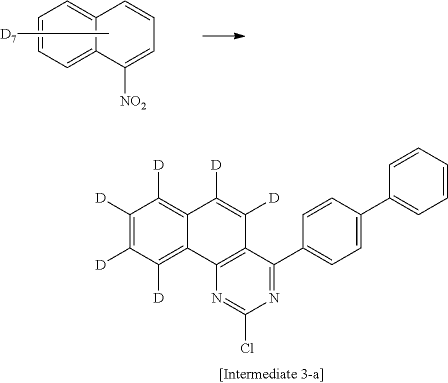

Intermediate 3-a (yield 48%) was synthesized in the same manner as in Reactions 1-1 to 1-3, except that heptadeuteronitronaphthalene and 4-biphenylmagnesium bromide were used instead of 1-nitronaphthalene in Reaction 1-1 and phenylmagnesium bromide in Reaction 1-2, respectively.

[Reaction 3-2] Synthesis of Intermediate 3-b

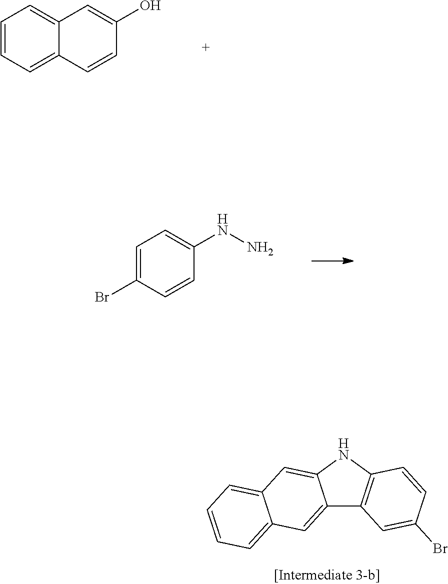 [SMILES: Oc1ccc2ccccc2c1 ; NNc1ccc(Br)cc1 ; Brc1ccc2[nH]c3cc4ccccc4cc3c2c1]

2-Naphthol (20 g, 0.14 mol), sodium bisulfite (28.8 g, 0.28 mol), and 4-bromophenylhydrazine (31.2 mL, 0.17 mol) were stirred in 160 mL of distilled water at 120° C. for 12 h. To the mixture was added an aqueous solution of hydrochloric acid. After stirring at 100° C. for about 1 h, the reaction mixture was extracted with dichloromethane and separated by column chromatography, affording Intermediate 3-b (yield 22%).

[Reaction 3-3] Synthesis of Intermediate 3-c

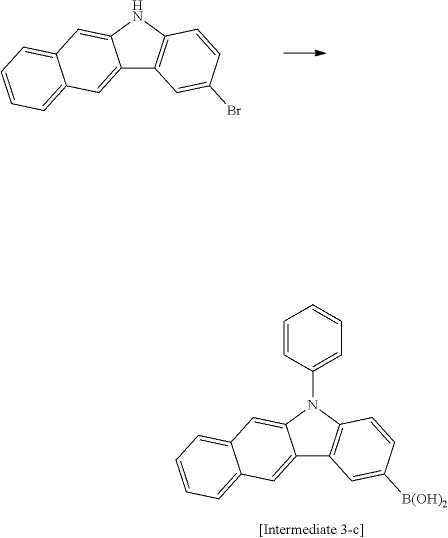 [SMILES: Brc1ccc2[nH]c3cc4ccccc4cc3c2c1 ; OB(O)c1ccc2c(c1)c1cc3ccccc3cc1n2-c1ccccc1]

Intermediate 3-c (yield 64%) was synthesized in the same manner as in Reactions 1-5 and 1-6, except that Intermediate 3-b synthesized in Reaction 3-2 was used instead of 4-bromo-9H-carbazole in Reaction 1-5.

[Reaction 3-4] Synthesis of Compound 26

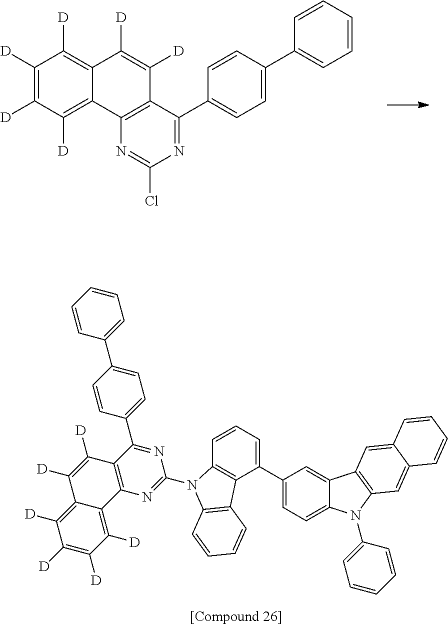 [SMILES: [2H]c1c([2H])c([2H])c2c(c1[2H])c([2H])c([2H])c1c(-c3ccc(-c4ccccc4)cc3)nc(Cl)nc12 ; [2H]c1c([2H])c([2H])c2c(c1[2H])c([2H])c([2H])c1c(-c3ccc(-c4ccccc4)cc3)nc(-n3c4ccccc4c4c(-c5ccc6c(c5)c5cc7ccccc7cc5n6-c5ccccc5)cccc43)nc12]

Compound 9 (yield 40%) was synthesized in the same manner as in Reactions 1-4 and 1-7, except that Intermediate 3-a synthesized in Reaction 3-1 and Intermediate 3-c synthesized in Reaction 3-3 were used instead of Intermediate 1-c in Reaction 1-4 and Intermediate 1-f in Reaction 1-7, respectively.

MS: m/z 795

[Synthesis Example 4] Synthesis of Compound 19

[Reaction 4-1] Synthesis of Intermediate 4-a

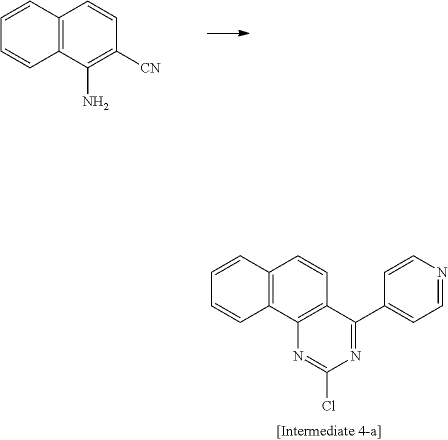 [SMILES: N#Cc1ccc2ccccc2c1N ; Clc1nc(-c2ccncc2)c2ccc3ccccc3c2n1]

Intermediate 4-a (yield 46%) was synthesized in the same manner as in Reactions 1-2 and 1-3, except that pyridin-4-ylmagnesium bromide was used instead of phenylmagnesium bromide in Reaction 1-2.

[Reaction 4-2] Synthesis of Compound 19

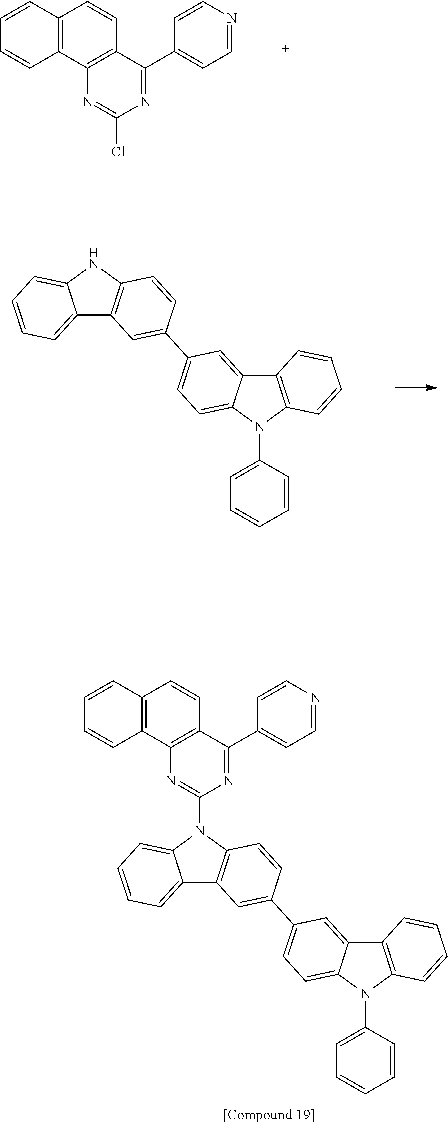 [SMILES: Clc1nc(-c2ccncc2)c2ccc3ccccc3c2n1 ; c1ccc(-n2c3ccccc3c3cc(-c4ccc5[nH]c6ccccc6c5c4)ccc32)cc1 ; c1ccc(-n2c3ccccc3c3cc(-c4ccc5c(c4)c4ccccc4n5-c4nc(-c5ccncc5)c5ccc6ccccc6c5n4)ccc32)cc1]

Compound 19 (yield 32%) was synthesized in the same manner as in Reaction 1-4, except that Intermediate 2-c synthesized in Reaction 2-3 and Intermediate 4-a synthesized in Reaction 4-1 were used instead of 4-bromo-9H-carbazole and Intermediate 1-c, respectively.

MS: m/z 664

### SYNTHESIS EXAMPLE 5

### Synthesis of Compound 21

[Reaction 5-1] Synthesis of Intermediate 5-a

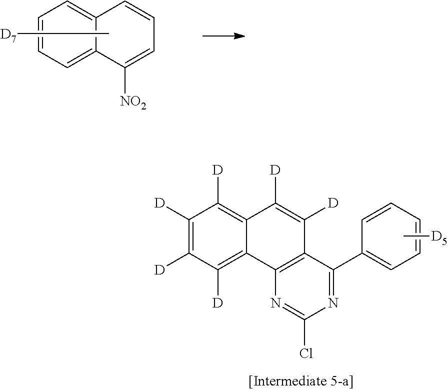

Intermediate 5-a (yield 45%) was synthesized in the same manner as in Reactions 1-1 to 1-3, except that heptadeuteronitronaphthalene and pentadeuterophenylmagnesium bromide were used instead of 2-nitronaphthalene in Reaction 1-1 and phenylmagnesium bromide in Reaction 1-2, respectively.

[Reaction 5-2] Synthesis of Intermediate 5-b

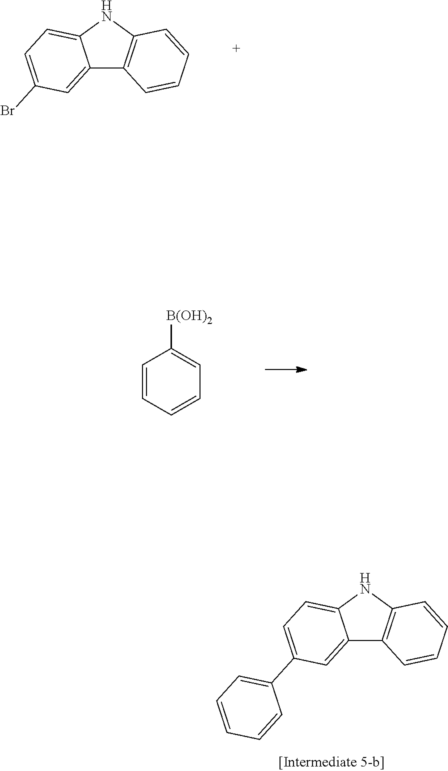 [SMILES: Brc1ccc2[nH]c3ccccc3c2c1 ; OB(O)c1ccccc1 ; c1ccc(-c2ccc3[nH]c4ccccc4c3c2)cc1]

Intermediate 5-b (yield 82%) was synthesized in the same manner as in Reaction 1-7, except that 3-bromo-9H-carbazole and phenylboronic acid were used instead of Intermediate 1-d and Intermediate 1-f, respectively.

[Reaction 5-3] Synthesis of Intermediate 5-c

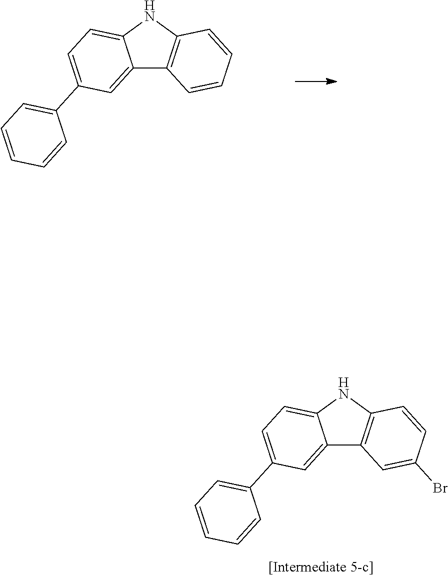 [SMILES: c1ccc(-c2ccc3[nH]c4ccccc4c3c2)cc1 ; Brc1ccc2[nH]c3ccc(-c4ccccc4)cc3c2c1]

Intermediate 5-b (35 g, 0.14 mol) synthesized in Reaction 5-2 was dissolved in 250 mL of dimethylformamide. Thereafter, the solution was stirred at 0° C. To the solution was added dropwise a solution of NBS (26.7 g, 0.15 mol) in 100 mL of dimethylformamide. The mixture was allowed to warm to room temperature, followed by stirring for 12 h. Distilled water was added dropwise to precipitate a solid. The solid was collected by filtration and recrystallized from toluene and methanol, affording 39.1 g of Intermediate 5-c (yield 86%).

[Reaction 5-4] Synthesis of Intermediate 5-d

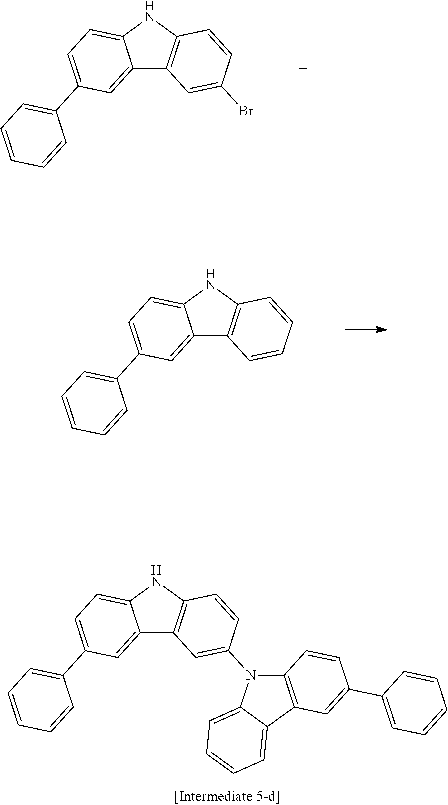 [SMILES: Brc1ccc2[nH]c3ccc(-c4ccccc4)cc3c2c1 ; c1ccc(-c2ccc3[nH]c4ccccc4c3c2)cc1 ; c1ccc(-c2ccc3[nH]c4ccc(-n5c6ccccc6c6cc(-c7ccccc7)ccc65)cc4c3c2)cc1]

Intermediate 5-d (yield 68%) was synthesized in the same manner as in Reaction 1-5, except that Intermediate 5-b synthesized in Reaction 5-2 and Intermediate 5-c synthesized in Reaction 5-3 were used instead of 4-bromo-9H-carbazole and iodobenzene, respectively.

[Reaction 5-5] Synthesis of Compound 21

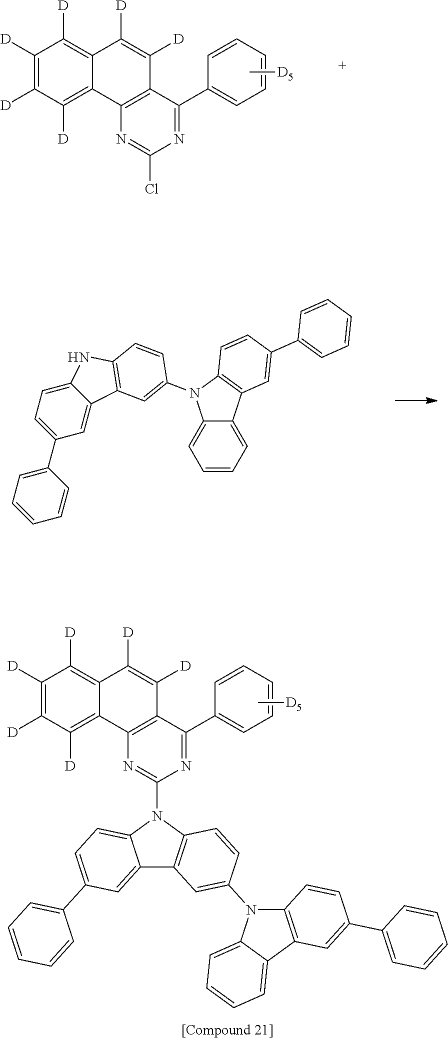

Compound 21 (yield 42%) was synthesized in the same manner as in Reaction 1-4, except that Intermediate 5-d synthesized in Reaction 5-4 and Intermediate 5-a synthesized in Reaction 5-1 were used instead of 4-bromo-9H-carbazole and Intermediate 1-c, respectively.

MS: m/z 770

### SYNTHESIS EXAMPLE 6

### Synthesis of Compound 24

[Reaction 6-1] Synthesis of Intermediate 6-a

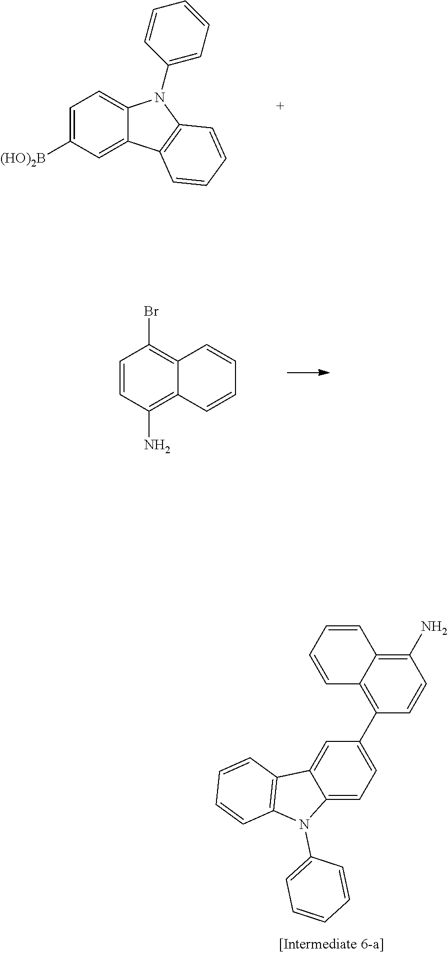 [SMILES: OB(O)c1ccc2c(c1)c1ccccc1n2-c1ccccc1 ; Nc1ccc(Br)c2ccccc12 ; Nc1ccc(-c2ccc3c(c2)c2ccccc2n3-c2ccccc2)c2ccccc12]

Intermediate 6-a (yield 81%) was synthesized in the same manner as in Reaction 1-7, except that 1-amino-4-bromonaphthalene and 9-phenyl-9H-carbazole-3-boronic acid were used instead of Intermediate 1-d and Intermediate 1-f, respectively.

[Reaction 6-2] Synthesis of Intermediate 6-b

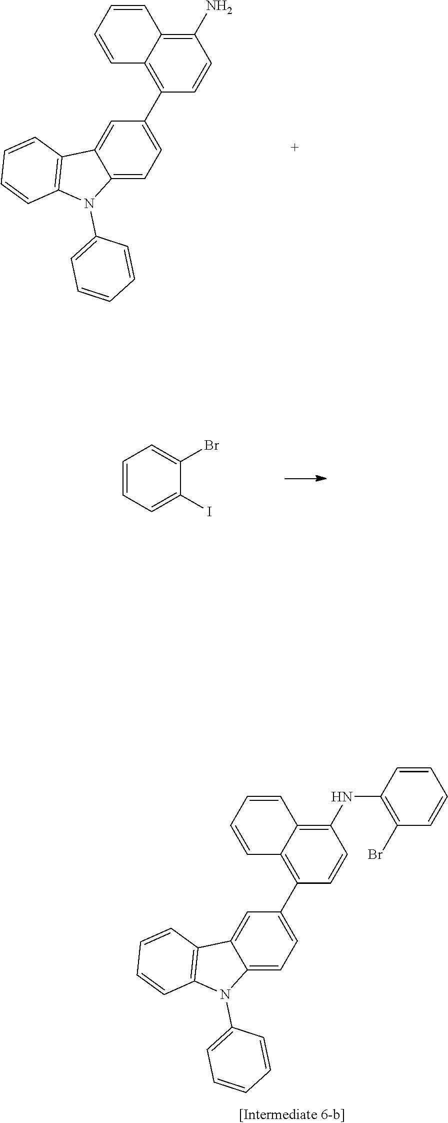 [SMILES: Nc1ccc(-c2ccc3c(c2)c2ccccc2n3-c2ccccc2)c2ccccc12 ; Brc1ccccc1I ; Brc1ccccc1Nc1ccc(-c2ccc3c(c2)c2ccccc2n3-c2ccccc2)c2ccccc12]

Intermediate 6-a (30.7 g, 0.08 mol) synthesized in Reaction 6-1, 2-bromoiodobenzene (22.6 g, 0.08 mol), tris(dibenzylideneacetone)dipalladium (1.1 g, 0.0001 mol), 1,1′-bis(diphenylphosphino)ferrocene (0.7 g, 0.0001 mol), and sodium tert-butoxide (15.3 g, 0.16 mol) were refluxed in 250 mL of toluene for 24 h. The reaction mixture was separated by column chromatography, affording 18.5 g of Intermediate 6-b (yield 43%).

[Reaction 6-3] Synthesis of Intermediate 6-c

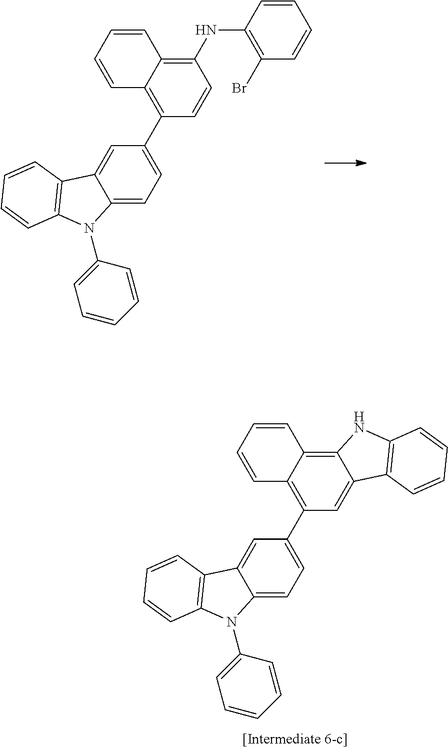 [SMILES: Brc1ccccc1Nc1ccc(-c2ccc3c(c2)c2ccccc2n3-c2ccccc2)c2ccccc12 ; c1ccc(-n2c3ccccc3c3cc(-c4cc5c6ccccc6[nH]c5c5ccccc45)ccc32)cc1]

Intermediate 6-b (16.2 g, 0.03 mol) synthesized in Reaction 6-2, potassium acetate (4.0 g, 0.04 mol), and tetrakis(triphenylphosphine)palladium (0.8 g, 0.001 mol) were stirred in 125 mL of dimethylformamide at 150° C. for 24 h. The reaction mixture was separated by column chromatography and recrystallized from hexane, affording 3.5 g of Intermediate 6-c (yield 25%).

[Reaction 6-4] Synthesis of Compound 24

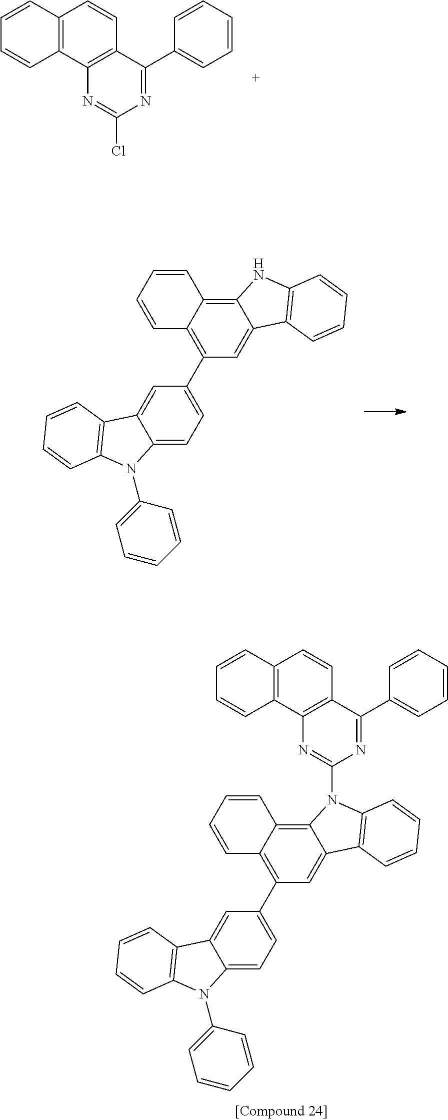 [SMILES: Clc1nc(-c2ccccc2)c2ccc3ccccc3c2n1 ; c1ccc(-n2c3ccccc3c3cc(-c4cc5c6ccccc6[nH]c5c5ccccc45)ccc32)cc1 ; c1ccc(-c2nc(-n3c4ccccc4c4cc(-c5ccc6c(c5)c5ccccc5n6-c5ccccc5)c5ccccc5c43)nc3c2ccc2ccccc23)cc1]

Compound 24 (yield 42%) was synthesized in the same manner as in Reaction 1-4, except that Intermediate 6-c synthesized in Reaction 6-3 was used instead of 4-bromo-9H-carbazole.

MS: m/z 713

### SYNTHESIS EXAMPLE 7

### Synthesis of Compound 6 [Reaction 7-1] Synthesis of Intermediate 7-a

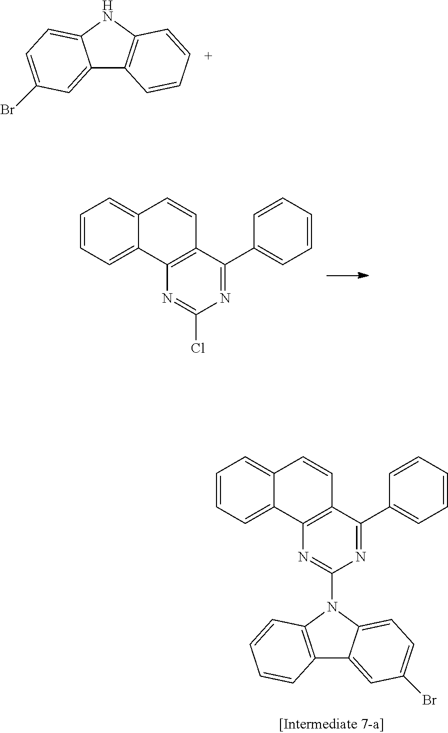 [SMILES: Brc1ccc2[nH]c3ccccc3c2c1 ; Clc1nc(-c2ccccc2)c2ccc3ccccc3c2n1 ; Brc1ccc2c(c1)c1ccccc1n2-c1nc(-c2ccccc2)c2ccc3ccccc3c2n1]

Intermediate 7-a (yield 80%) was synthesized in the same manner as in Reaction 1-4, except that 3-bromo-9H-carbazole was used instead of 4-bromo-9H-carbazole.

[Reaction 7-2] Synthesis of Intermediate 7-b

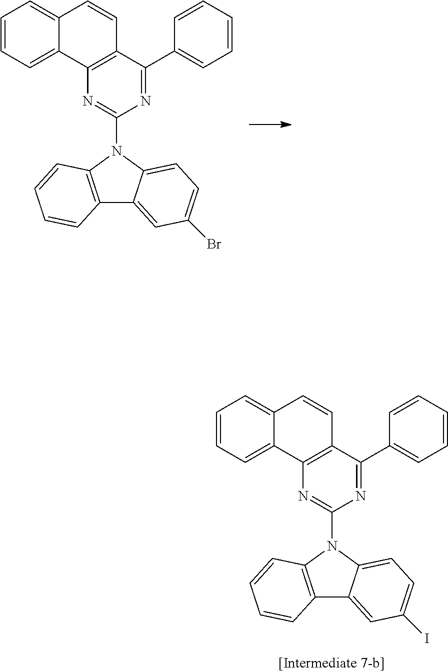 [SMILES: Brc1ccc2c(c1)c1ccccc1n2-c1nc(-c2ccccc2)c2ccc3ccccc3c2n1 ; Ic1ccc2c(c1)c1ccccc1n2-c1nc(-c2ccccc2)c2ccc3ccccc3c2n1]

Intermediate 7-a (50.0 g, 0.10 mol) synthesized in Reaction 7-1 was added to 250 mL of tetrahydrofuran. After cooling to −78° C., 1.6 M n-butyllithium (61 mL, 0.10 mol) was slowly added dropwise. The mixture was stirred for 1 h and then iodine (26.5 g, 0.10 mol) was slowly added thereto. The resulting mixture was allowed to warm to room temperature. After stirring for 2 h, an aqueous solution of sodium thiosulfate was added. The reaction mixture was extracted with ethyl acetate, and then the organic layer was concentrated under reduced pressure and recrystallized from hexane, affording 43.8 g of Intermediate 7-b (yield 80%).

[Reaction 7-3] Synthesis of Intermediate 7-c

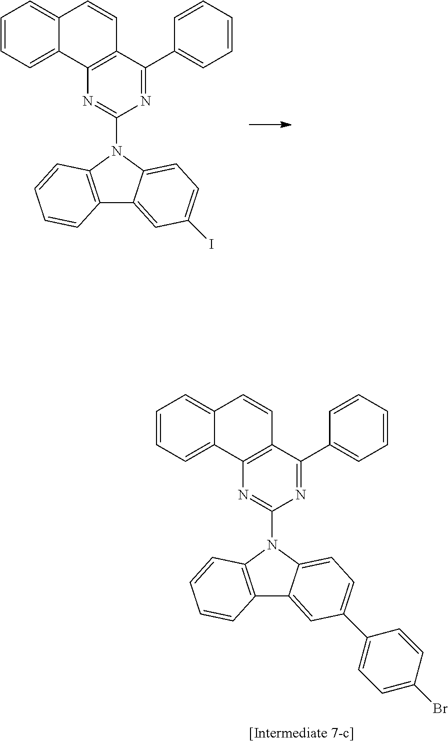 [SMILES: Ic1ccc2c(c1)c1ccccc1n2-c1nc(-c2ccccc2)c2ccc3ccccc3c2n1 ; Brc1ccc(-c2ccc3c(c2)c2ccccc2n3-c2nc(-c3ccccc3)c3ccc4ccccc4c3n2)cc1]

Intermediate 7-c (yield 68%) was synthesized in the same manner as in Reaction 1-7, except that Intermediate 7-b synthesized in Reaction 7-2 and 4-bromophenylboronic acid were used instead of Intermediate 1-d and Intermediate 1-f, respectively.

[Reaction 7-4] Synthesis of Compound 6

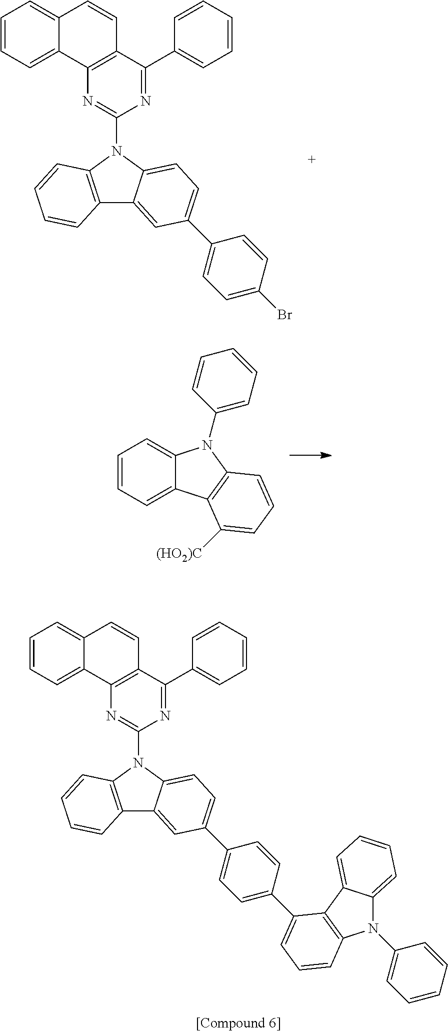

Compound 13 (yield 70%) was synthesized in the same manner as in Reaction 1-7, except that Intermediate 13-c synthesized in Reaction 13-3 and Intermediate 1-f synthesized in Reaction 1-6 were used instead of Intermediate 1-d and Intermediate 1-f, respectively.

MS: m/z 739

### SYNTHESIS EXAMPLE 8

### Synthesis of Compound 13

[Reaction 8-1] Synthesis of Intermediate 8-a

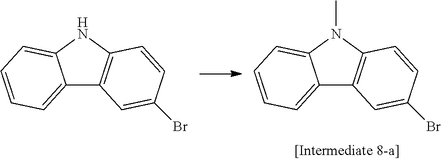

5.85 g (244 mmol) of sodium hydride and 150 mL of dimethylformamide were mixed and stirred under a nitrogen atmosphere. After cooling to 0° C., a solution of 30 g (122 mmol) of 3-bromo-9H-carbazole in 250 mL of dimethylformamide was added dropwise. The mixture was allowed to warm to room temperature, followed by stirring for 2 h. After cooling to 0° C., a solution of 38.05 g (146 mmol) of iodomethane in 50 mL of dimethylformamide was added dropwise. The resulting mixture was allowed to warm to room temperature, followed by stirring for 12 h. The reaction mixture was extracted with ethyl acetate and separated by column chromatography, affording 30.0 g of Intermediate 8-a (yield 97%).

[Reaction 8-2] Synthesis of Intermediate 8-b

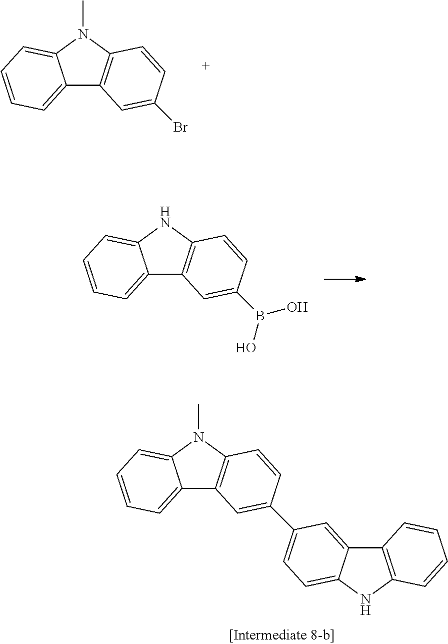 [SMILES: Cn1c2ccccc2c2cc(Br)ccc21 ; OB(O)c1ccc2[nH]c3ccccc3c2c1 ; Cn1c2ccccc2c2cc(-c3ccc4[nH]c5ccccc5c4c3)ccc21]

19.2 g of Intermediate 8-b (yield 65%) was synthesized in the same manner as in Reaction 1-7, except that Intermediate 8-a synthesized in Reaction 8-1 and 9H-carbazol-3-ylboronic acid were used instead of Intermediate 1-d and Intermediate 1-f, respectively.

[Reaction 8-3] Synthesis of Intermediate 8-c

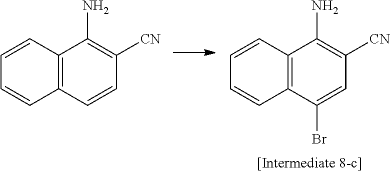 [SMILES: N#Cc1ccc2ccccc2c1N ; N#Cc1cc(Br)c2ccccc2c1N]

Intermediate 1-a (50.0 g, 297 mmol) synthesized in Reaction 1-1 and 50 mL of dimethylformamide were stirred. The mixture was cooled to 0° C. and a solution of 55.56 g (312 mmol) of NBS in 250 mL of dimethylformamide was added dropwise thereto. The resulting mixture was allowed to warm to room temperature, followed by stirring for additional 4 h. Distilled water was added to precipitate a solid. The solid was collected by filtration and purified by column chromatography, affording 68 g of Intermediate 8-c (yield 93%).

[Reaction 8-4] Synthesis of Intermediate 8-d

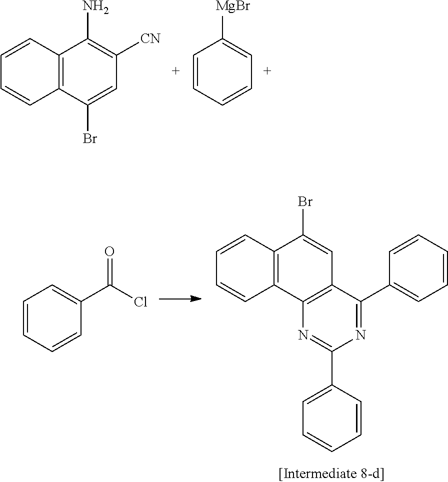

Intermediate 8-d (yield 63%) was synthesized in the same manner as in Reaction 1-2, except that Intermediate 8-c synthesized in Reaction 8-3 and benzoyl chloride were used instead of Intermediate 1-a and ethyl chloroformate, respectively.

[Reaction 8-5] Synthesis of Compound 13

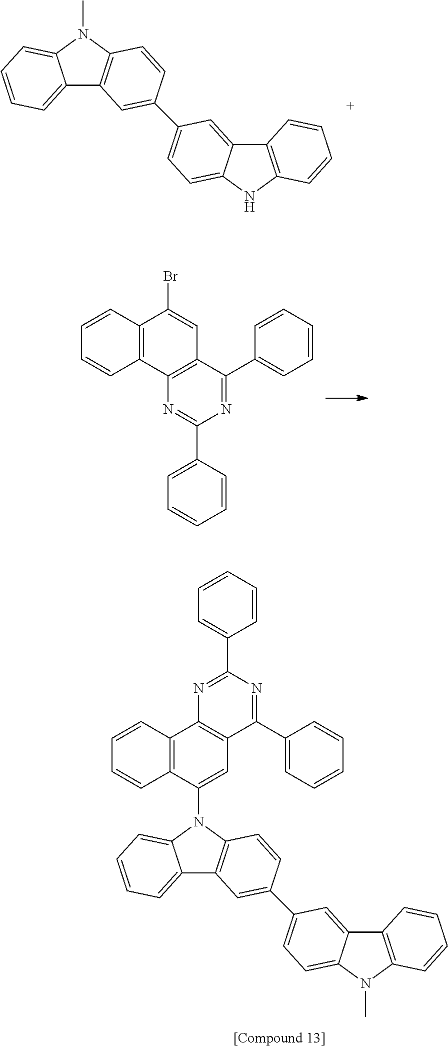 [SMILES: Cn1c2ccccc2c2cc(-c3ccc4[nH]c5ccccc5c4c3)ccc21 ; Brc1cc2c(-c3ccccc3)nc(-c3ccccc3)nc2c2ccccc12 ; Cn1c2ccccc2c2cc(-c3ccc4c(c3)c3ccccc3n4-c3cc4c(-c5ccccc5)nc(-c5ccccc5)nc4c4ccccc34)ccc21]

Compound 13 (yield 31%) was synthesized in the same manner as in Reaction 1-5, except that Intermediate 8-b synthesized in Reaction 8-2 and Intermediate 8-d synthesized in Reaction 8-4 were used instead of 4-bromo-9H-carbazole and iodobenzene, respectively.

MS: m/z 677

### EXAMPLES 1-8

### Fabrication of Organic Light Emitting Diodes

ITO glass was patterned to have a light emitting area of 2 mm×2 mm, followed by cleaning. After the cleaned ITO glass was mounted in a vacuum chamber, the base pressure was adjusted to 1×10⁻⁶ torr. DNTPD (700 Å), α-NPD (300 Å), Compound 2, 8, 26, 19, 21, 24, 6 or 13+RD-1 (10%) (300 Å), and Compound A:Liq=1:1 (250 Å), Liq (10 Å), and Al (1,000 Å) were sequentially deposited on the ITO to form organic layers, completing the fabrication of an organic light emitting diode. The luminescent properties of the organic light emitting diode were measured at 0.4 mA.

The structures of the DNTPD, NPD, RD-1, Compound A, and Liq are as follows:

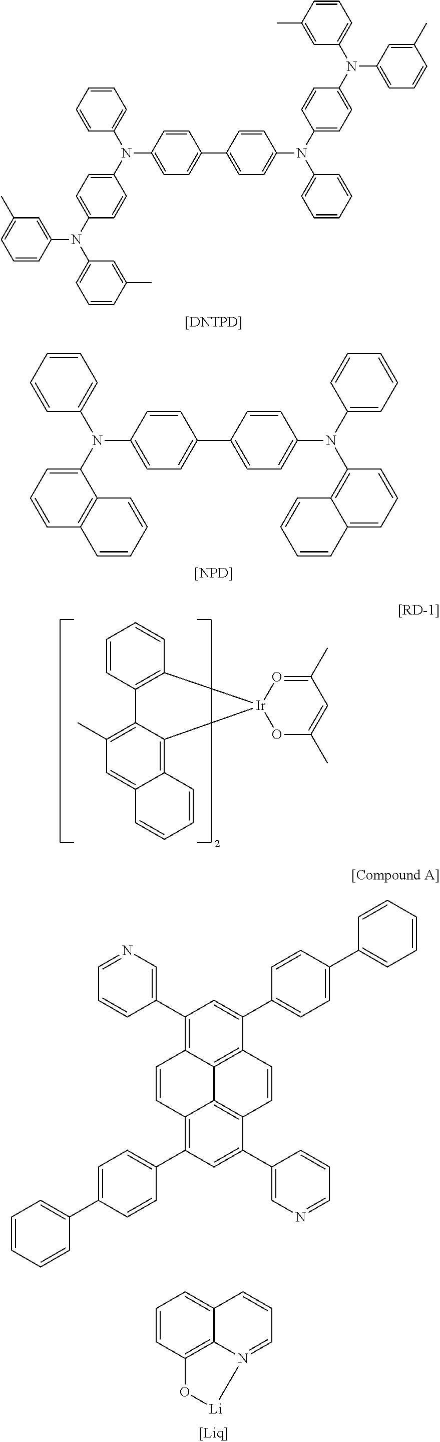

### COMPARATIVE EXAMPLES 1 AND 2

An organic light emitting diode was fabricated in the same manner as in Examples 1-8, except that BAlq (Comparative Example 1) or Compound B (Comparative Example 2) was used instead of the inventive organic compounds. BAlq and Compound B are phosphorescent host materials well known in the art and their structures are as follows:

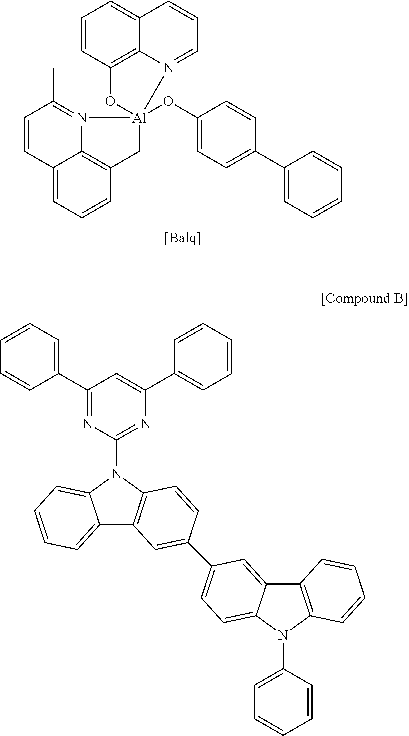

The organic electroluminescence devices fabricated in Examples 1-8 and Comparative Example 1-2 were measured for voltage, current density, luminance, color coordinates, and life. The results are shown in Table 1. T₉₅ indicates the time at which the luminance of each device was decreased to 95% of the initial luminance (3000 cd/m²).

TABLE 1

| | | Doping | Current | Luminance | | | Life |
|---|---|---|---|---|---|---|---|
| Example No. | Host | concentration (%) | density (V) | (Cd/m²) | CIEx | CIEy | (T₉₅, hr) |
| Comparative | BAlq | 10 | 6.2 | 1470 | 0.665 | 0.334 | 40 |
| Example1 | | | | | | | |
| Comparative | Compound B | 10 | 4.0 | 1520 | 0.663 | 0.335 | 50 |
| Example2 | | | | | | | |
| Example 1 | 2 | 10 | 4.5 | 1630 | 0.664 | 0.334 | 270 |
| Example2 | 8 | 10 | 4.3 | 2100 | 0.665 | 0.334 | 430 |
| Example3 | 26 | 10 | 4.6 | 1600 | 0.665 | 0.334 | 320 |
| Example4 | 19 | 10 | 4.0 | 2000 | 0.666 | 0.333 | 400 |
| Example5 | 21 | 10 | 4.1 | 1800 | 0.665 | 0.334 | 290 |
| Example6 | 24 | 10 | 3.9 | 1700 | 0.664 | 0.335 | 250 |
| Example7 | 6 | 10 | 4.2 | 1830 | 0.664 | 0.335 | 230 |
| Example8 | 13 | 10 | 3.8 | 2000 | 0.665 | 0.334 | 330 |

As can be seen from the results in Table 1, the inventive organic compounds had much lower driving voltages than BAlq, which is widely known as a phosphorescent host material, and had higher luminous efficiencies and longer lives than BAlq and Compound B.

### INDUSTRIAL APPLICABILITY

The organic electroluminescence devices employing the organic light emitting compounds according to the present invention can be driven at low voltages compared to conventional devices employing phosphorescent host materials. The low-voltage driving leads to high power efficiency while at the same time achieving improved luminous efficiency and life characteristics. Therefore, the organic electroluminescence devices of the present invention are suitable for use in systems selected from flat panel displays, flexible displays, monochromatic flat panel lighting systems, white flat panel lighting systems, flexible monochromatic lighting systems, and flexible white lighting systems.

1. An organic light emitting compound represented by Formula 1: 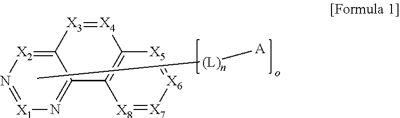
wherein X₁ to X₈ are identical to or different from each other and are each independently N or CRₒ, provided that when CRₒ exists in plurality, the CRₒ groups are identical to or different from each other, and A is represented by Formula A1 or A2: 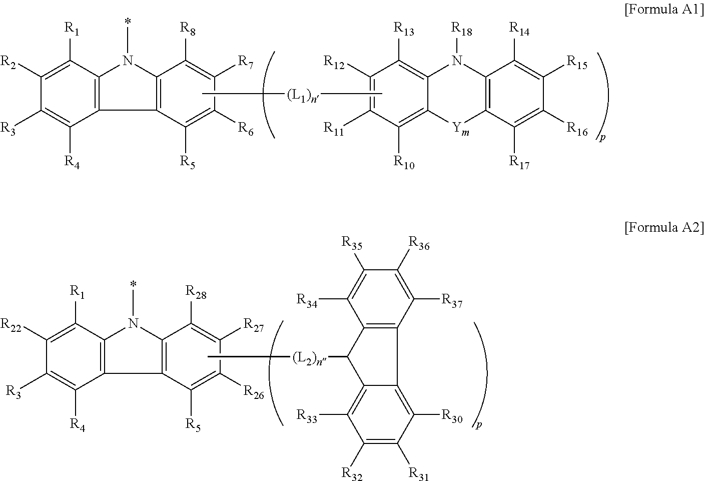
wherein each asterisk (*) represents a site at which the nitrogen atom is bonded to L, Rₒ, R₁ to R₈, R₁₀ to R₁₈, R₂₁ to R₂₈, and R₃₀ to R₃₇ are identical to or different from each other and are each independently selected from a hydrogen atom, a deuterium atom, substituted or unsubstituted C₁-C₃₀ alkyl groups, substituted or unsubstituted C₆-C₄₀ aryl groups, substituted or unsubstituted C₂-C₃₀ heteroaryl groups, halogen atoms, a hydroxyl group, a cyano group, a nitro group, an amino group, an amidino group, a hydrazino group, a hydrazono group, a carboxyl group or salts thereof, a sulfonic acid group or salts thereof, a phosphoric acid group or salts thereof, substituted or unsubstituted C₂-C₆₀ alkenyl groups, substituted or unsubstituted C₂-C₆₀ alkynyl groups, substituted or unsubstituted C₁-C₆₀ alkoxy groups, substituted or unsubstituted C₃-C₆₀ cycloalkyl groups, substituted or unsubstituted C₅-C₃₀ cycloalkenyl groups, substituted or unsubstituted C₅-C₆₀ aryloxy groups, substituted or unsubstituted C₁-C₃₀ alkylthioxy groups, substituted or unsubstituted C₅-C₃₀ arylthioxy groups, substituted or unsubstituted C₅-C₆₀ arylthio groups, —SiR₃₈R₃₉R₄₀, and —NR₄₁R₄₂, Rₒ, R₁ to R₈, R₁₀ to R₁₈, R₂₁ to R₂₈, R₃₀ to R₃₇, and substituents thereof are optionally bonded together to form a saturated or unsaturated ring, Y is a single bond, CR₅₁R₅₂, NR₅₃, O, S, Se, SiR₅₄R₅₅, GeR₅₆R₅₇, PR₅₈, PR₅₉(═O), C═O or BR₆₀, m is an integer of 1 or 2, provided that when m is 2, the plurality of Y groups are identical to or different from each other, L, L₁, and L₂ are each independently a single bond or are each independently selected from substituted or unsubstituted C₁-C₆₀ alkylene groups, substituted or unsubstituted C₂-C₆₀ alkenylene groups, substituted or unsubstituted C₂-C₆₀ alkynylene groups, substituted or unsubstituted C₃-C₆₀ cycloalkylene groups, substituted or unsubstituted C₂-C₆₀ heterocycloalkylene groups, substituted or unsubstituted C₅-C₆₀ arylene groups, and substituted or unsubstituted C₂-C₆₀ heteroarylene groups, with the proviso that L, L₁, and L₂ are each independently optionally combined with an adjacent substituent to form a saturated or unsaturated ring, n, n′, and n″ are each independently an integer from 0 to 3, provided that when n, n′, and n″ are equal to or greater than 2, the plurality of L, L₁, and L₂ groups are identical to or different from each other, respectively, o and p are each independently an integer from 1 to 3, and R₃₈ to R₄₂ and R₅₁ to R₆₀ have the same meanings as Rₒ, R₁ to R₈, R₁₀ to R₁₈, R₂₁ to R₂₈, and R₃₀ to R₃₇.

2. The organic light emitting compound according to claim 1, wherein L, L₁, and L₂ are each independently selected from the following structures: 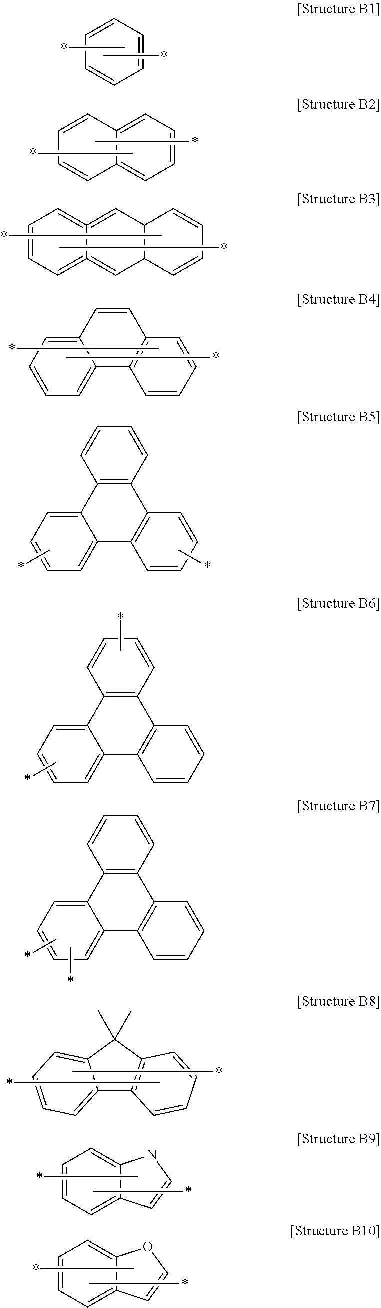 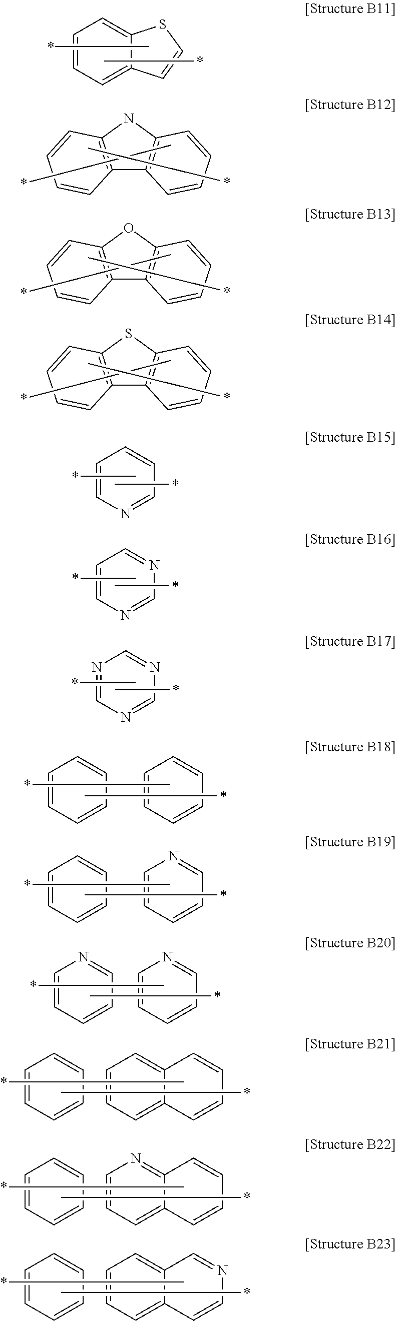 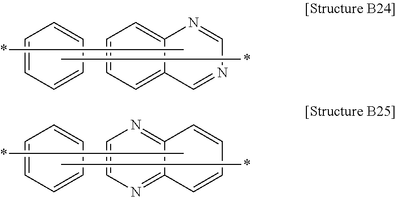
wherein hydrogen or deuterium atoms are optionally bonded to the carbon atoms of the aromatic rings and R optionally replaces the nitrogen atoms, R having the same meaning as R₁ to R₈, R₁₀ to R₁₈, R₂₁ to R₂₈, and R₃₀ to R₃₇ defined in Formula 1.

3. The organic light emitting compound according to claim 1, wherein the compound of Formula 1 is selected from compounds represented by Formulae 2 to 8: 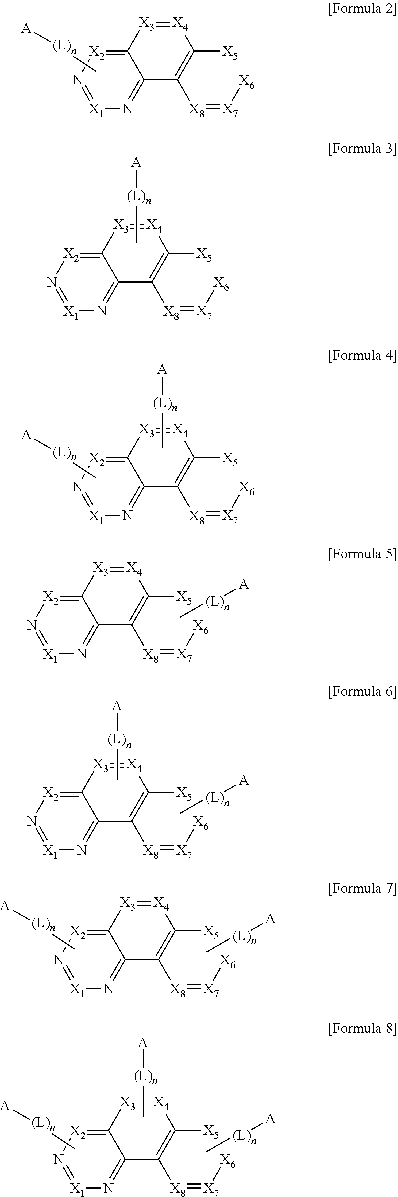
wherein L, X₁ to X₈, A, and n are as defined in Formula 1.

4. The organic light emitting compound according to claim 1, wherein the compound of Formula 1 is selected from the following compounds 1 to 29:

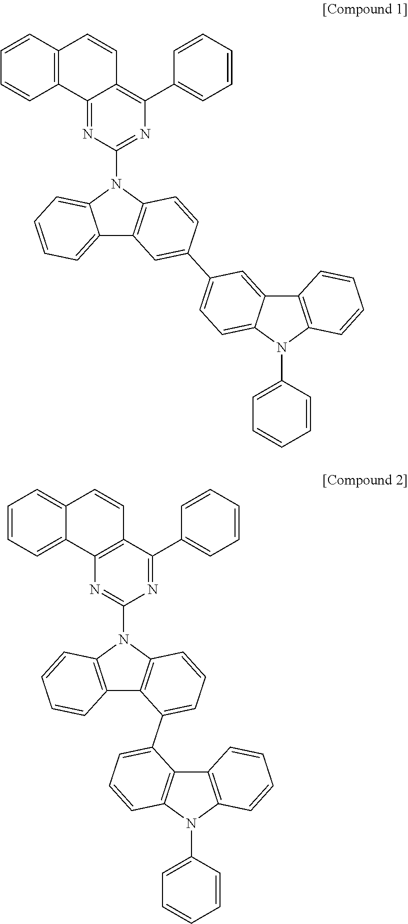 [SMILES: c1ccc(-c2nc(-n3c4ccccc4c4cc(-c5ccc6c(c5)c5ccccc5n6-c5ccccc5)ccc43)nc3c2ccc2ccccc23)cc1 ; c1ccc(-c2nc(-n3c4ccccc4c4c(-c5cccc6c5c5ccccc5n6-c5ccccc5)cccc43)nc3c2ccc2ccccc23)cc1]

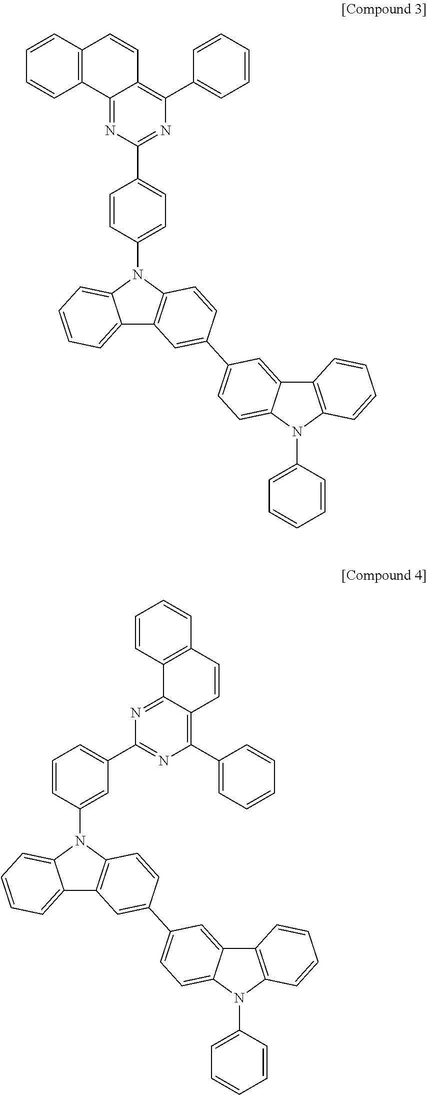 [SMILES: c1ccc(-c2nc(-c3ccc(-n4c5ccccc5c5cc(-c6ccc7c(c6)c6ccccc6n7-c6ccccc6)ccc54)cc3)nc3c2ccc2ccccc23)cc1 ; c1ccc(-c2nc(-c3cccc(-n4c5ccccc5c5cc(-c6ccc7c(c6)c6ccccc6n7-c6ccccc6)ccc54)c3)nc3c2ccc2ccccc23)cc1]

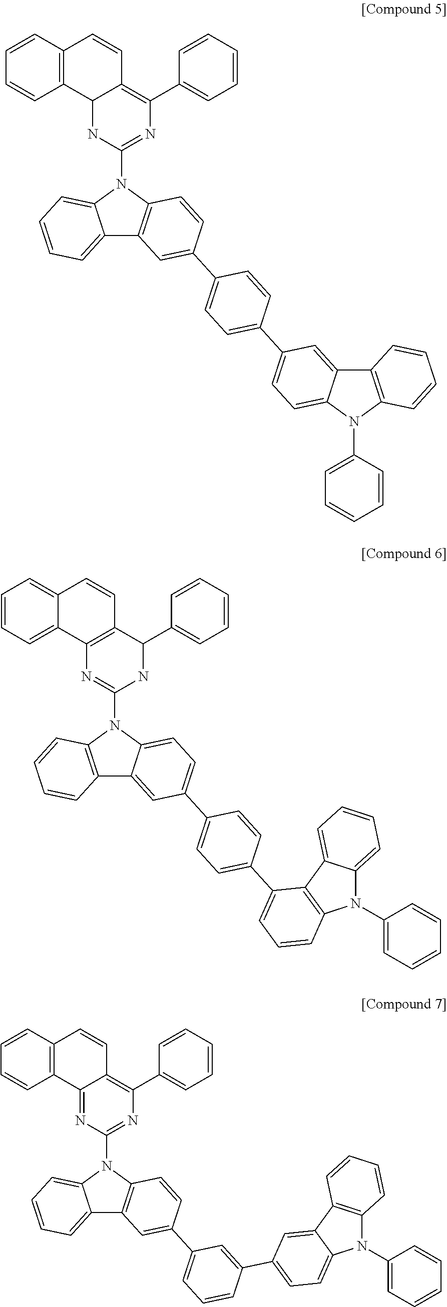 [SMILES: C1=Cc2ccccc2C2NC(n3c4ccccc4c4cc(-c5ccc(-c6ccc7c(c6)c6ccccc6n7-c6ccccc6)cc5)ccc43)=NC(c3ccccc3)=C12 ; c1ccc(C2NC(n3c4ccccc4c4cc(-c5ccc(-c6cccc7c6c6ccccc6n7-c6ccccc6)cc5)ccc43)=Nc3c2ccc2ccccc32)cc1 ; c1ccc(-c2nc(-n3c4ccccc4c4cc(-c5cccc(-c6ccc7c(c6)c6ccccc6n7-c6ccccc6)c5)ccc43)nc3c2ccc2ccccc23)cc1]

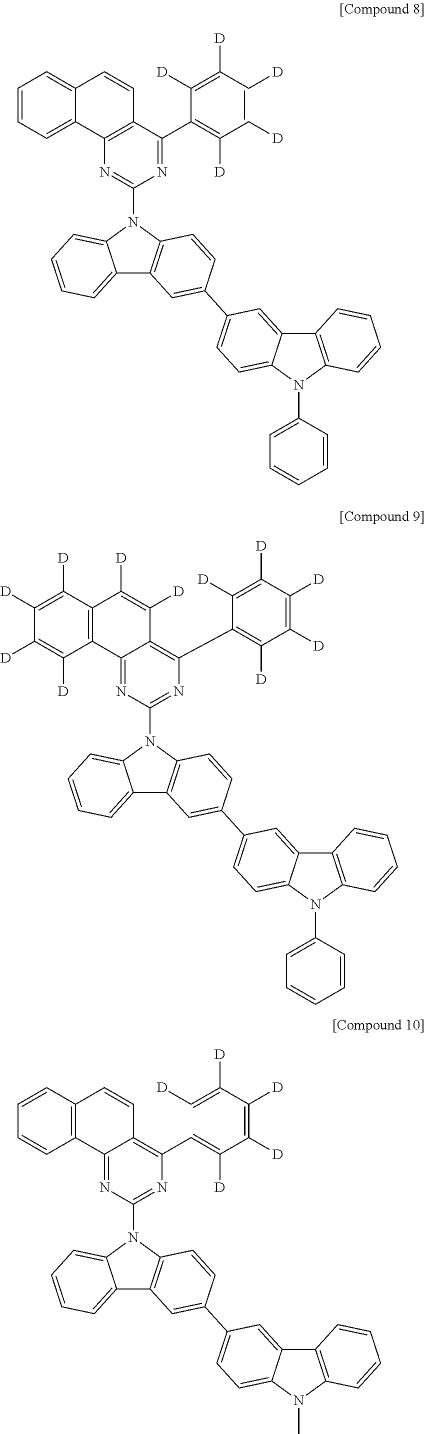 [SMILES: [2H]C/C([2H])=C([2H])\C(=C(\[2H])C[2H])c1nc(-n2c3ccccc3c3cc(-c4ccc5c(c4)c4ccccc4n5-c4ccccc4)ccc32)nc2c1ccc1ccccc12 ; [2H]c1c([2H])c([2H])c(-c2nc(-n3c4ccccc4c4cc(-c5ccc6c(c5)c5ccccc5n6-c5ccccc5)ccc43)nc3c2c([2H])c([2H])c2c([2H])c([2H])c([2H])c([2H])c23)c([2H])c1[2H] ; [2H]/C=C([2H])/C([2H])=C([2H])\C([2H])=C\c1nc(-n2c3ccccc3c3cc(-c4ccc5c(c4)c4ccccc4n5C)ccc32)nc2c1ccc1ccccc12]

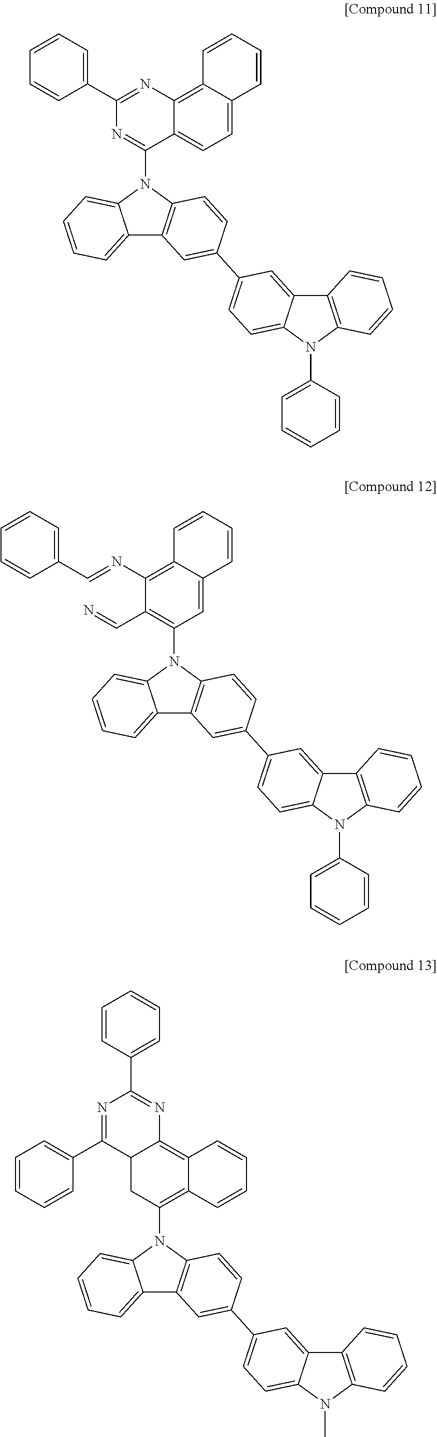 [SMILES: c1ccc(-c2nc(-n3c4ccccc4c4cc(-c5ccc6c(c5)c5ccccc5n6-c5ccccc5)ccc43)c3ccc4ccccc4c3n2)cc1 ; N=Cc1c(-n2c3ccccc3c3cc(-c4ccc5c(c4)c4ccccc4n5-c4ccccc4)ccc32)cc2ccccc2c1/N=C/c1ccccc1 ; Cn1c2ccccc2c2cc(-c3ccc4c(c3)c3ccccc3n4C3=c4ccccc4=C4N=C(c5ccccc5)N=C(c5ccccc5)C4C3)ccc21]

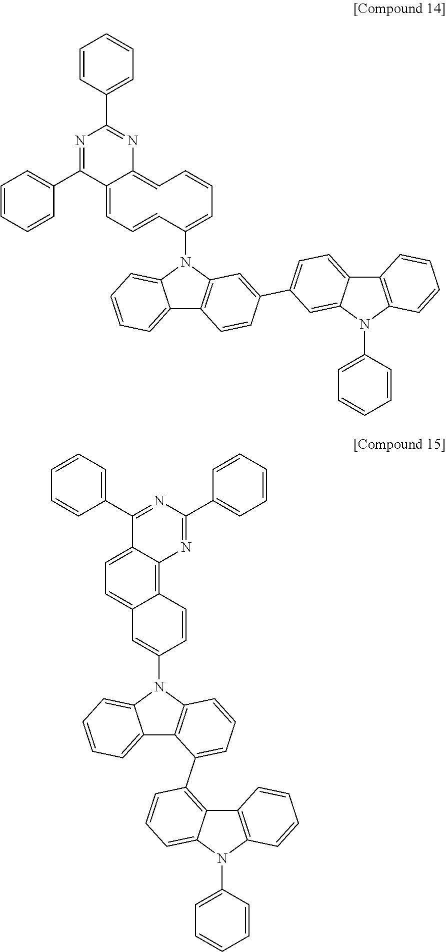 [SMILES: c1ccc(-c2nc(-c3ccccc3)c3cccc(-n4c5ccccc5c5ccc(-c6ccc7c8ccccc8n(-c8ccccc8)c7c6)cc54)ccccc3n2)cc1 ; c1ccc(-c2nc(-c3ccccc3)c3ccc4cc(-n5c6ccccc6c6c(-c7cccc8c7c7ccccc7n8-c7ccccc7)cccc65)ccc4c3n2)cc1]

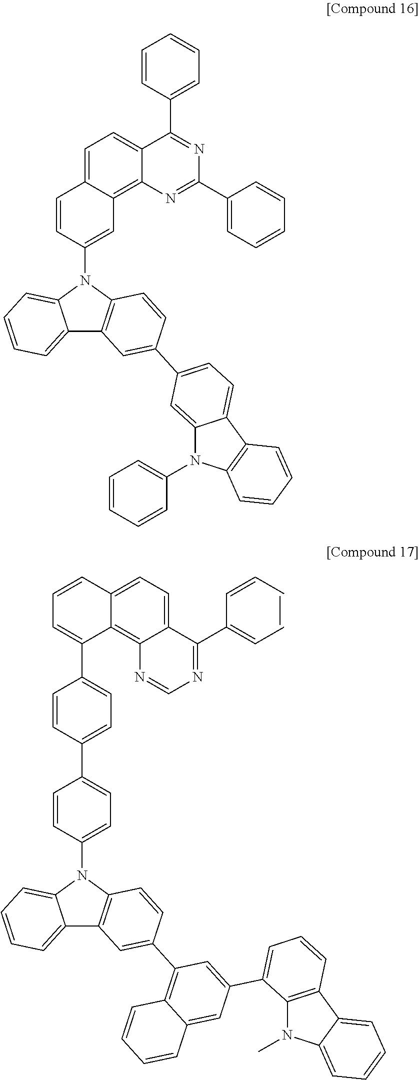 [SMILES: c1ccc(-c2nc(-c3ccccc3)c3ccc4ccc(-n5c6ccccc6c6cc(-c7ccc8c9ccccc9n(-c9ccccc9)c8c7)ccc65)cc4c3n2)cc1 ; C/C=C\C(=C/C)c1ncnc2c1ccc1cccc(-c3ccc(-c4ccc(-n5c6ccccc6c6cc(-c7cc(-c8cccc9c%10ccccc%10n(C)c89)cc8ccccc78)ccc65)cc4)cc3)c12]

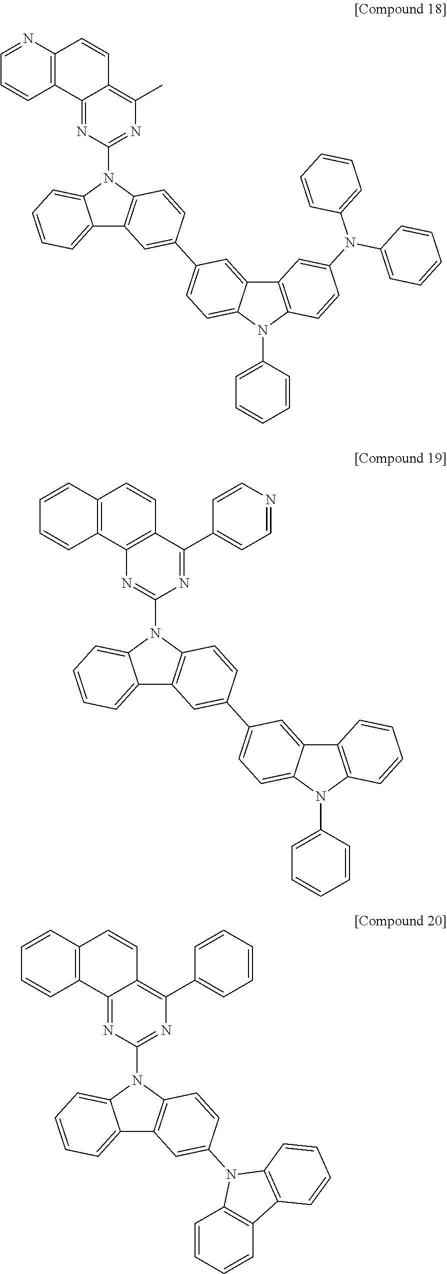 [SMILES: Cc1nc(-n2c3ccccc3c3cc(-c4ccc5c(c4)c4cc(N(c6ccccc6)c6ccccc6)ccc4n5-c4ccccc4)ccc32)nc2c1ccc1ncccc12 ; c1ccc(-n2c3ccccc3c3cc(-c4ccc5c(c4)c4ccccc4n5-c4nc(-c5ccncc5)c5ccc6ccccc6c5n4)ccc32)cc1 ; c1ccc(-c2nc(-n3c4ccccc4c4cc(-n5c6ccccc6c6ccccc65)ccc43)nc3c2ccc2ccccc23)cc1]

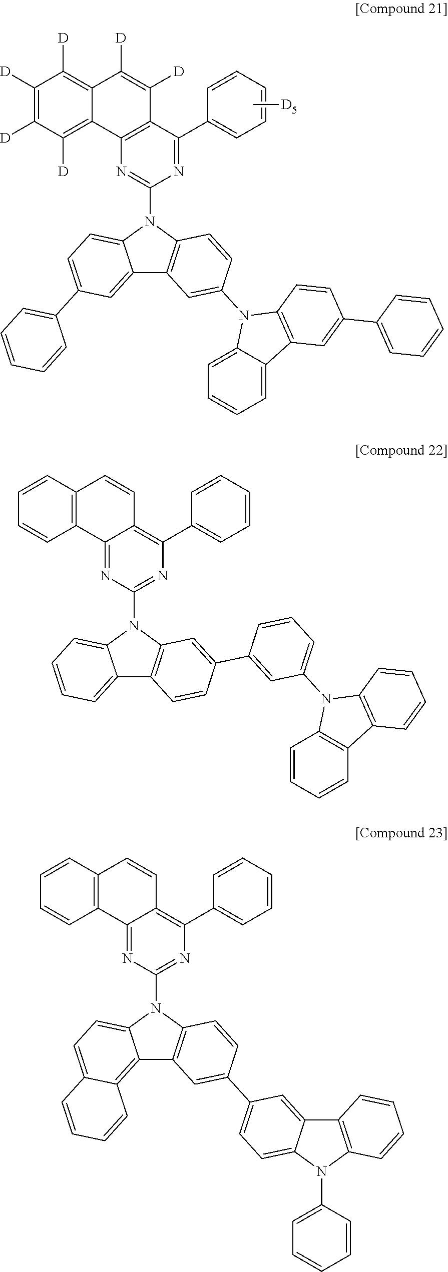

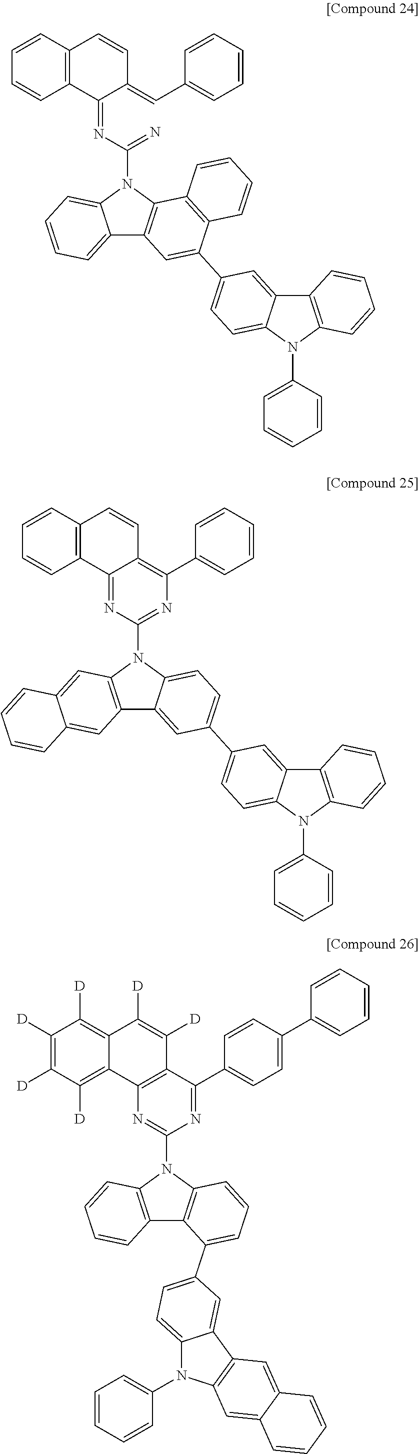 [SMILES: N=C(/N=C1\C(=C\c2ccccc2)C=Cc2ccccc21)n1c2ccccc2c2cc(-c3ccc4c(c3)c3ccccc3n4-c3ccccc3)c3ccccc3c21 ; c1ccc(-c2nc(-n3c4ccc(-c5ccc6c(c5)c5ccccc5n6-c5ccccc5)cc4c4cc5ccccc5cc43)nc3c2ccc2ccccc23)cc1 ; [2H]c1c([2H])c([2H])c2c(c1[2H])c([2H])c([2H])c1c(-c3ccc(-c4ccccc4)cc3)nc(-n3c4ccccc4c4c(-c5ccc6c(c5)c5cc7ccccc7cc5n6-c5ccccc5)cccc43)nc12]

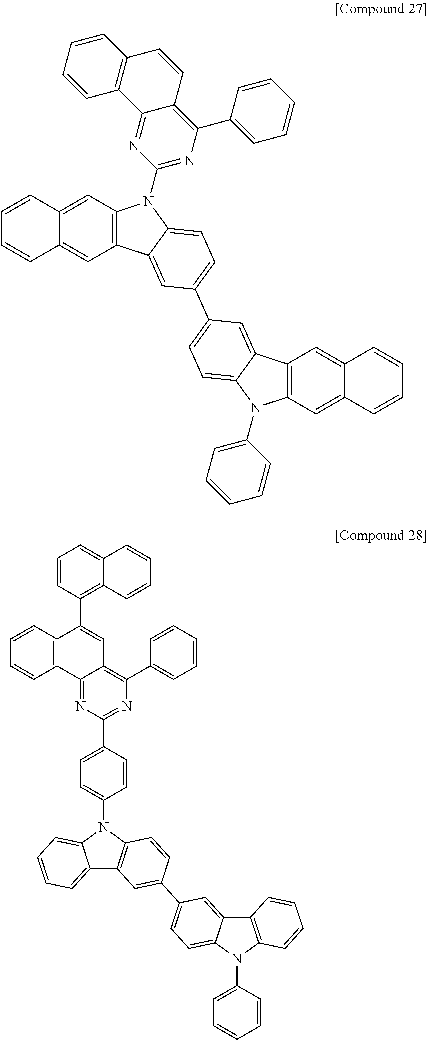 [SMILES: c1ccc(-c2nc(-n3c4ccc(-c5ccc6c(c5)c5cc7ccccc7cc5n6-c5ccccc5)cc4c4cc5ccccc5cc43)nc3c2ccc2ccccc23)cc1 ; C1=C\C/C(c2cccc3ccccc23)=C\c2c(nc(-c3ccc(-n4c5ccccc5c5cc(-c6ccc7c(c6)c6ccccc6n7-c6ccccc6)ccc54)cc3)nc2-c2ccccc2)C\C=C/1]

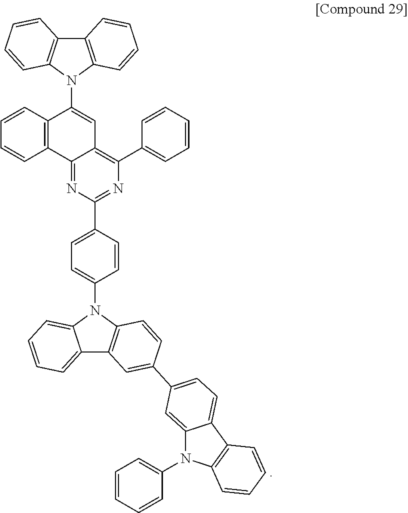 [SMILES: c1ccc(-c2nc(-c3ccc(-n4c5ccccc5c5cc(-c6ccc7c8ccccc8n(-c8ccccc8)c7c6)ccc54)cc3)nc3c2cc(-n2c4ccccc4c4ccccc42)c2ccccc23)cc1]

5. An organic electroluminescence device comprising a first electrode, a second electrode, and at least one organic layer interposed between the first and second electrodes wherein the organic layer comprises the organic light emitting compound of Formula 1 according to claim 1.

6. The organic electroluminescence device according to claim 5, wherein the organic layer comprises at least one layer selected from a hole injecting layer, a hole transport layer, a functional layer having functions of both hole injection and hole transport, a light emitting layer, an electron transport layer, an electron injecting layer, and a layer having functions of both electron transport and electron injection.

7. The organic electroluminescence device according to claim 5, wherein the organic layer interposed between the first and second electrodes comprises a light emitting layer.

8. The organic electroluminescence device according to claim 5, wherein the light emitting layer is composed of at least one host compound and at least one dopant compound and the host compound comprises the organic light emitting compound of Formula 1 according to claim 1.

9. The organic electroluminescence device according to claim 8, wherein the dopant compound comprises at least one compound selected from copper, boron, iridium, platinum, palladium, and ruthenium complexes.

10. The organic electroluminescence device according to claim 5, wherein the organic layer is formed in plurality and the organic layers are each independently formed by a monomolecular deposition or solution process.

11. The organic electroluminescence device according to claim 5, wherein the organic layer further comprises one or more organic red, green or blue light emitting layers to achieve white light emission.

12. The organic electroluminescence device according to claim 5, wherein the organic electroluminescence device is used in a system selected from flat panel displays, flexible displays, monochromatic flat panel lighting systems, white flat panel lighting systems, flexible monochromatic lighting systems, and flexible white lighting systems.
